(19) United States
(12) Patent Application Publication
(10) Pub. No.: US 2011/0118239 A1
(43) Pub. Date: May 19, 2011

(54) PREPARATION AND ENANTIOMERIC SEPARATION OF 7-(3-PYRIDINYL)-1,7-DIAZASPIRO[4.4]NONANE AND NOVEL SALT FORMS OF THE RACEMATE AND ENANTIOMERS

(75) Inventors: Jessica Beaver, Lewisville, NC (US); Scott Breining, Winston-Salem, NC (US); Gary Maurice Dull, Lewisville, NC (US); Gregory J. Gatto, Winston-Salem, NC (US); John Genus, Winston-Salen, NC (US); Jacob Mathew, Winston-Salem, NC (US); Julio A. Munoz, Walnut Grove, NC (US); Inigo Pfeiffer, Kernersville, NC (US); Steve M. Ttoler, Winston-Salen, NC (US); James Wamsley, Pfafftown, NC (US); Jianxun Xie, Clemmons, NC (US)

(73) Assignee: Targacept, Inc., Winston-Salem, NC (US)

(21) Appl. No.: 12/812,647

(22) PCT Filed: Jan. 15, 2009
(86) PCT No.: PCT/US2009/000242

§ 371 Date: Jan. 21, 2011

Related U.S. Application Data
(60) Provisional application No. 61/021,197, filed on Jan. 15, 2008.

(51) Int. Cl. A61K 31/551; C07D 223/32; A61P 25/18; A61P 25/22; A61P 25/24; A61P 25/28

(52) U.S. Cl. 514/221; 540/543

(57) ABSTRACT

A novel scalable synthesis for the preparation of 7-(3-pyridinyI)-1,7-diazaspiro[4.4)nonane has been developed, and 7-(3-pyridinyl)-1,7-diazaspiro[4.4]nonane salts have been formed with succinic acid and oxalic acid. Additionally, 7-(3-pyridinyl)-1,7-diaza-spiro[4.4]nonane has been separated into its stereoisomers via resolution with L and D di-p-toluoyltartaric acids, giving (R)- and (S)-7-(3-pyridinyl)-1,7-diazaspiro[4.4]nonane of high enantiomeric purity. Numerous solid salts of the resulting (R)- and (S)-7-(3-pyridinyl)-1,7-diazaspiro[4.4}nonane have been prepared. Methods for the preparation of the racemic and enantiomeric salts, pharmaceutical compositions comprising such salts, and uses thereof are disclosed. The salts can be administered to patients susceptible to or suffering from conditions and disorders, such as central nervous system disorders, to treat and/or prevent such disorders.

### FIELD OF THE INVENTION

The present invention relates to a method for the preparation of 7-(3-pyridinyl)-1,7-diazaspiro[4.4]nonane, (R)-7-(3-pyridinyl)-1,7-diazaspiro[4,4]nonane, and (S)-7-(3-pyridinyl)-1,7-diazaspiro[4.4]nonane and to novel salt forms of these compounds, as well as pharmaceutical compositions comprising the salts. Additionally, the present invention involves methods for treating a wide variety of conditions and disorders, and particularly conditions and disorders associated with dysfunction of the central and autonomic nervous systems, and more particularly conditions and disorders which can be treated by modulation of neuronal nicotinic receptors (NNRs), using the novel salt forms.

### BACKGROUND OF THE INVENTION

The therapeutic potential of compounds that target neuronal nicotinic receptors (NNRs), also known as nicotinic acetylcholine receptors (nAChRs), has been the subject of several reviews (see, for example, Breining et al., Ann. Rep. Med. Chem. 40: 3 (2005), Hogg and Bertrand, Curr. Drug Targets: CNS Neurol. Disord. 3: 123 (2004), Suto and Zacharias, Expert Opin. Ther. Targets 8: 61 (2004), Dani et al., Bioorg. Med. Chem. Lett. 14: 1837 (2004), Bencherif and Schmitt, Curr. Drug Targets: CNS Neurol. Disord. 1: 349 (2002)). Among the kinds of indications for which NNR ligands have been proposed as therapies are cognitive disorders, including Alzheimer's disease, attention deficit disorder, and schizophrenia (Newhouse et al., Curr. Opin. Pharmacol. 4: 36 (2004), Levin and Rezvani, Curr. Drug Targets: CNS Neurol. Disord. 1: 423 (2002), Graham et al., Curr. Drug Targets: CNS Neurol. Disord. 1: 387 (2002), Ripoll et al., Curr. Med. Res. Opin. 20(7): 1057 (2004), and McEvoy and Allen, Curr. Drug Targets: CNS Neural. Disord. 1: 433 (2002)); pain and inflammation (Decker et al., Curr. Top. Med. Chem. 4(3): 369 (2004), Vincler, Expert Opin. Invest. Drugs 14(10): 1191 (2005), Jain, Curr. Opin. Inv. Drugs 5: 76 (2004), Miao et al., Neuroscience 123: 777 (2004)); depression and anxiety (Shytle et al., Mol. Psychiatry. 7: 525 (2002), Damaj et al., Mol. Pharmacol. 66: 675 (2004), Shytle et al., Depress. Anxiety 16: 89 (2002)); neurodegeneration (O'Neill et al., Curr. Drug Targets: CNS Neurol. Disord. 1: 399 (2002), Takata et al., J. Pharmacol. Exp. Ther. 306: 772 (2003), Marrero et al., J. Pharmacol. Exp. Ther. 309: 16 (2004)); Parkinson's disease (Jonnala and Buccafusco, J. Neurosci. Res. 66: 565 (2001)); addiction (Dwoskin and Crooks, Biochem. Pharmacol. 63: 89 (2002), Coe et al., Bioorg. Med. Chem. Lett. 15(22): 4889 (2005)); obesity (Li et al., Curr. Top. Med. Chem. 3: 899 (2003)); and Tourette's syndrome (Sacco et al., J. Psychopharmacol. 18(4): 457 (2004), Young et al., Clin. Ther. 23(4): 532 (2001)).

There exists a heterogeneous distribution of nAChR subtypes in both the central and peripheral nervous systems. For instance, the nAChR subtypes which are predominant in vertebrate brain are α4β2, α7, and α3β2, whereas those which predominate at the autonomic ganglia are α3β4 and those of neuromuscular junction are α1β1γδ and α1β1γε (see Dwoskin et al., Exp. Opin. Ther. Patents 10: 1561 (2000) and Holliday et al. J. Med. Chem. 40(26), 4169 (1997)).

A limitation of some nicotinic compounds is that they are associated with various undesirable side effects due to non-specific binding to multiple nAChR subtypes. For example, binding to and stimulation of muscle and ganglionic nAChR subtypes can lead to side effects which can limit the utility of a particular nicotinic binding compound as a therapeutic agent. Such side effects include significant increases in blood pressure and heart rate, significant negative effects upon the gastro-intestinal tract, and significant effects upon skeletal muscle.

The compound 7-(3-pyridinyl)-1,7-diazaspiro[4.4]nonane is a neuronal nicotinic receptor (NNR) modulator with selectivity for the α4β2 nicotinic subtype over other nicotinic subtypes, for example, the α7 subtype, the ganglionic, and the muscle subtypes.

The compound 7-(3-pyridinyl)-1,7-diazaspiro[4.4]nonane or a salt thereof is believed to provide benefits in the treatment or prevention of central nervous system (CNS) disorders. The compound, its synthesis, and its use in methods of medical treatment, is described, for example, in U.S. Pat. Nos. 6,956,042 and 7,291,731, and in U.S. application Ser. Nos. 11/207,102 and 12/042,778, the contents of which are hereby incorporated by reference.

The commercial development of a drug candidate, such as 7-(3-pyridinyl)-1,7-diazaspiro[4.4]nonane, involves many steps, including the development of a cost effective synthetic method that is adaptable to a large scale manufacturing process. Commercial development also involves research regarding salt forms of the drug substance that exhibit suitable purity, chemical stability, pharmaceutical properties, and characteristics that facilitate convenient handling and processing. Furthermore, compositions containing the drug substance should have adequate shelf life. That is, they should not exhibit significant changes in physicochemical characteristics such as, but not limited to, chemical composition, water content, density, hygroscopicity, stability, and solubility upon storage over an appreciable period of time. Additionally, reproducible and constant plasma concentration profiles of drug upon administration to a patient are also important factors.

Solid salt forms are generally preferred for oral formulations due to their tendency to exhibit these properties in a preferential way; and in the case of basic drugs such as racemic 7-(3-pyridinyl)-1,7-diazaspiro[4.4]nonane, or a single enantiomer thereof, acid addition salts are often the preferred salt form. However, different salt forms vary greatly in their ability to impart these properties and such properties cannot be predicted with reasonable accuracy. For example, some salts are solids at ambient temperatures, while other salts are liquids, viscous oils, or gums at ambient temperatures. Furthermore, some salt forms are stable to heat and light under extreme conditions and others readily decompose under much milder conditions. Thus, the development of a suitable acid addition salt form of a basic drug for use in a pharmaceutical composition is a highly unpredictable process.

Additionally, it is often beneficial to resolve racemic compounds, like 7-(3-pyridinyl)-1,7-diazaspiro[4.4]nonane, into their individual enantiomers, as each of the enantiomers may exhibit a unique set of pharmacological and toxicological properties, as compared to those of the other enantiomer and those of the racemate. Separation of 7-(3-pyridinyl)-1,7-diazaspiro[4.4]nonane into its enantiomers was disclosed in U.S. Pat. No. 6,956,042, but the methods disclosed therein (i.e., using a chiral acid to convert the enantiomeric mixture into diastereomeric amides, chromatographic separation of the amides, and chemical cleavage of the amides to obtain the enantiomeric amines) are characterized by low yields and variable product purity and are not amenable to large scale synthesis. Furthermore, U.S. Pat. No. 6,956,042 does not characterize the enantiomers as to either absolute stereochemistry or their pharmacology and toxicology. An enantiomeric separation amenable to commercial scale synthesis (i.e., one that involves fewer steps and no chromatography, and results in high purity compounds and high overall yields) would be highly advantageous. Additionally, it is necessary to characterize the individual enantiomers of 7-(3-pyridinyl)-1,7-diazaspiro[4.4]nonane, in terms of both their absolute stereochemistry and their pharmacology and toxicology, to determine if and how the enantiomers (and the racemate) may differ in terms of therapeutic benefit for various conditions and disorders.

### SUMMARY OF THE INVENTION

The present invention includes a synthesis of 7-(3-pyridinyl)-1,7-diazaspiro[4,4]nonane, producing a product of sufficient purity and quality for use in pharmaceutical compositions. The present invention also includes a method for the synthesis of 7-(3-pyridinyl)-1,7-diazaspiro[4.4]nonane suitable for large scale manufacture. Further, the present invention includes a method for manufacture of 7-(3-pyridinyl)-1,7-diazaspiro[4.4]nonane or a pharmaceutically acceptable salt thereof that is scalable to commercial manufacture. The invention also includes pharmaceutically acceptable salts, such as the succinic acid salt, of 7-(3-pyridinyl)-1,7-diazaspiro[4.4]nonane and methods of preparation of these salts.

The invention includes a scalable procedure for the separation of 7-(3-pyridinyl)-1,7-diazaspiro[4.4]nonane into its stereoisomers, (R)- and (S)-7-(3-pyridinyl)-1,7-diazaspiro[4,4]nonane, via resolution with (−)-di-O,O′-p-toluoyl-L-tartaric acid or (+)-di-O,O′-p-toluoyl-D-tartaric acid. The resolution involves efficient fractional crystallization and requires no chromatography.

The present invention also includes pharmaceutically acceptable salts, such as the benzoic acid, p-hydroxybenzoic acid, mandelic acid, hydrochloric acid, and mucic (galactaric) acid salts of (R)- and (S)-7-(3-pyridinyl)-1,7-diazaspiro[4.4]nonane, and also methods of preparation of these salts.

(R)-7-(3-Pyridinyl)-1,7-diazaspiro[4.4]nonane and (S)-7-(3-pyridinyl)-1,7-diazaspiro[4.4]nonane and their pharmaceutically acceptable salts, when employed in effective amounts, are believed to modulate the activity of the α4β2 NNRs without appreciable interaction with the α7 NNR subtype or the nicotinic subtypes that characterize the human ganglia or skeletal muscle. Hence, these compounds are believed capable of treating or preventing diseases, disorders and conditions without eliciting significant side effects associated with activity at ganglionic and neuromuscular sites. Such side effects include significant increases in blood pressure and heart rate, significant negative effects upon the gastro-intestinal tract, and significant effects upon skeletal muscle.

The present invention includes pharmaceutical compositions comprising (R)-7-(3-pyridinyl)-1,7-diazaspiro[4.4]nonane and (S)-7-(3-pyridinyl)-1,7-diazaspiro[4.4]nonane or pharmaceutically acceptable salts thereof. The pharmaceutical compositions of the present invention can be used for treating or preventing a wide variety of conditions or disorders, including those disorders characterized by dysfunction of nicotinic cholinergic neurotransmission or the degeneration of the nicotinic cholinergic neurons. The pharmaceutical compositions are believed to be safe and effective with regards to prevention and treatment of a wide variety of conditions and disorders.

The present invention includes methods for treating or preventing disorders and conditions, such as CNS disorders, mood disorders, addictions, inflammation, inflammatory response associated with bacterial and/or viral infection, pain, metabolic syndrome, autoimmune disorders, or other disorders described in further detail herein. The methods involve administering to a subject a therapeutically effective amount of a compound of the present invention or a pharmaceutically acceptable salt thereof or a pharmaceutical composition comprising the compounds.

Additionally, the present invention includes compounds that have utility as diagnostic agents and in receptor binding studies as described herein.

One aspect of the present invention includes an acid salt of 7-(3-pyridinyl)-1,7-diazaspiro[4.4]nonane, wherein the acid is succinic acid or oxalic acid. In one embodiment, the stoichiometry (molar ratio) of 7-(3-pyridinyl)-1,7-diazaspiro[4.4]nonane to the acid is between 1:2 and 2:1. In one embodiment, the stoichiometry (molar ratio) of 7-(3-pyridinyl)-1,7-diazaspiro[4.4]nonane to the acid is 1:1.

One aspect of the present invention is an acid salt of (R)-7-(3-pyridinyl)-1,7-diazaspiro[4.4]nonane, wherein the acid is hydrochloric acid, oxalic acid, (R)-mandelic acid, benzoic acid, p-bromobenzoic acid, p-hydroxybenzoic acid, galactaric (mucic) acid, or (+)-di-O,O′-p-toluoyl-D-tartaric acid. Another aspect of the present invention is an acid salt of (S)-7-(3-pyridinyl)-1,7-diazaspiro[4.4]nonane, wherein the acid is hydrochloric acid, oxalic acid, (S)-mandelic acid, benzoic acid, p-bromobenzoic acid, p-hydroxybenzoic acid, galactaric (mucic) acid, or (−)-di-O,O′-p-toluoyl-L-tartaric acid.

In one embodiment, the stoichiometry (molar ratio) of the isomer of 7-(3-pyridinyl)-1,7-diazaspiro[4.4]nonane to the acid is between 1:2 and 2:1. In one embodiment, the stoichiometry (molar ratio) of the isomer of 7-(3-pyridinyl)-1,7-diazaspiro[4.4]nonane to the acid is 1:1. One embodiment includes the p-hydroxybenzoic acid salt.

One aspect of the present invention includes (R)-7-(3-pyridinyl)-1,7-diazaspiro[4.4]nonane mono-p-hydroxybenzoate. Another aspect of the present invention includes (S)-7-(3-pyridinyl)-1,7-diazaspiro[4.4]nonane mono-p-hydroxybenzoate.

One aspect of the present invention includes (R)-7-(3-pyridinyl)-1,7-diazaspiro[4.4]nonane or a salt thereof substantially free of (S)-7-(3-pyridinyl)-1,7-diazaspiro[4.4]nonane or a salt thereof.

One aspect of the present invention includes an acid salt of (R)-7-(3-pyridinyl)-1,7-diazaspiro[4.4]nonane in substantially crystalline form.

One aspect of the present invention includes a pharmaceutical composition comprising a compound of the present invention, along with one or more pharmaceutically acceptable carrier.

One aspect of the present invention includes a method for treating or preventing a CNS disorder comprising administering to a subject in need thereof an effective amount of a compound of the present invention. One aspect of the present invention includes we of a compound of the present invention in the manufacture of a medicament for the treatment or prevention of a CNS disorder. One aspect of the present invention includes a compound of the present invention for use in treating or preventing a CNS disorder. In one embodiment, the disorder is selected from the group consisting of mania, anxiety, depression, panic disorders, bipolar disorders, generalized anxiety disorder, obsessive-compulsive disorder, rage outbursts, autism and Tourette's syndrome. In one embodiment, the disorder is selected from the group consisting of pre-senile dementia (early onset Alzheimer's disease), senile dementia (dementia of the Alzheimer's type), Alzheimer's disease, Lewy Body dementia, vascular dementia, AIDS dementia complex, HIV-dementia, Parkinsonism including Parkinson's disease, Pick's disease, progressive supranuclear palsy, Huntington's chorea, tardive dyskinesia, hyperkinesia, Creutzfeld-Jakob disease, epilepsy, attention deficit disorder, attention deficit hyperactivity disorder, dyslexia, schizophrenia, schizophreniform disorder, schizoaffective disorder, mild cognitive impairment (MCI), and age-associated memory impairment (AAMI). In one embodiment, the disorder is substance addiction.

One aspect of the present invention includes a method for treating or preventing pain or inflammation comprising administering to a subject in need thereof an effective amount of a compound of the present invention. One aspect of the present invention includes use of a compound of the present invention in the manufacture of a medicament for the treatment or prevention of pain or inflammation. One aspect of the present invention includes a compound of the present invention for use in treating or preventing pain or inflammation.

One aspect of the present invention includes a method of preparation of 7-(3-pyridinyl)-1,7-diazaspiro[4.4]nonane, comprising: i) successive reaction of an alkyl 1-benzoylpyrrolidine-2-carboxylate with a strong base to form an enolate, and bromoacetonitrile, ii) sequential reduction of the resulting alkyl 1-benzoyl-2-cyanomethylpyrrolidine-2-carboxylate, first with hydrogen over palladium on carbon, and then with a metal hydride reagent, iii) palladium-catalyzed condensation of the resulting 1-benzyl-1,7-diazaspiro[4.4]nonane with 3-bromopyridine, and iv) removal of the benzyl group by hydrogenation over wet palladium on carbon; as well as products formed from such process.

One aspect of the present invention includes a method of separating isomers of 7-(3-pyridinyl)-1,7-diazaspiro[4.4]nonane comprising: (i) converting into diastereomeric salts by reaction with one or both of the stereoisomers of a chiral acid, (ii) isolating the individual diastereomeric salts by fractional crystallization, and (iii) liberating the free bases from the isolated salts by treatment with base; as well as products formed from such process. In one embodiment, the chiral acid is one or both of (+)-di-O,O′-p-toluoyl-D-tartaric acid and (−)-di-O,O′-p-toluoyl-L-tartaric acid.

One aspect of the present invention includes a method for preparation of (R)- and (S)-7-(3-pyridinyl)-1,7-diazaspiro[4.4]nonane in substantially pure enantiomeric form comprising: (i) conversion of a suitably N-protected racemic 2-allylproline into a pair of diastereomeric amides by condensation with a pure enantiomer of an amine containing a chiral auxiliary, (ii) separation of the diastereomers by means of either chromatography or crystallization, and (iii) completion of the synthesis in such a manner as the chiral auxiliary is cleaved. In one embodiment, the pair of diastereomeric intermediates is the N-benzoyl-2-allylproline (R)-α-methylbenzyl amides.

The scope of the present invention relates to combinations of aspects, embodiments, and preferences.

The foregoing and other aspects of the present invention are explained in further detail in the detailed description and examples set forth below.

### BRIEF DESCRIPTION OF THE FIGURES

FIG. 1 is a graphical illustration of the anxiolytic-like effects exhibited by Compound A, (R)-7-(3-pyridinyl)-1,7-diazaspiro[4.4]nonane, in a rat elevated plus maze test.

FIG. 2 is a graphical illustration of the effectiveness of Compound A, (R)-7-(3-pyridinyl)-1,7-diazaspiro[4.4]nonane, in the tail suspension model of depression in mice.

FIG. 3 graphically illustrates the mean (SD) terminal elimination half-life data for Compound A, (R)-7-(3-pyridinyl)-1,7-diazaspiro[4.4]nonane; Compound B, (S)-7-(3-pyridinyl)-1,7-diazaspiro[4.4]nonane; and Compound C racemic 7-(3-pyridinyl)-1,7-diazaspiro[4.4]nonane.

FIG. 4 is a comparison of the calculated XRPDs for the two crystalline forms of (R)-7-(3-pyridinyl)-1,7-diazaspiro[4.4]nonane p-chlorobenzoate.

FIGS. 5 and 6 are three-dimensional images of the two molecules of (R)-7-(3-pyridinyl)-1,7-diazaspiro[4.4]nonane p-chlorobenzoate in the asymmetric unit cell.

### DETAILED DESCRIPTION

### Definitions

The following definitions are meant to clarify, but not limit, the terms defined. If a particular term used herein is not specifically defined, such term should not be considered indefinite. Rather, terms are used within their accepted meanings.

As used herein, the term “compound(s)” may be used to mean the free base form, or alternatively, a salt form of 7-(3-pyridinyl)-1,7-diazaspiro[4.4]nonane, or an isomer thereof, depending on the context, which will be readily apparent. Those skilled in the art will be able to distinguish the difference.

As used herein, the phrase “pharmaceutically acceptable” refers to carrier(s), diluent(s), excipient(s) or salt forms of the compound of Formula I that are compatible with the other ingredients of the composition and not deleterious to the recipient of the pharmaceutical composition.

As used herein, the phrase “pharmaceutical grade” refers to a compound or composition of a standard suitable for use as a medicine. With reference to the discussion herein, pharmaceutical grade compounds of the present invention, particularly salt forms thereof, display appropriate properties, including purity, stability, solubility, and bioavailability for use in a drug product. Preferential characteristics include those that would increase the ease or efficiency of manufacture of the active ingredient and its composition into a commercial drug product. Furthermore, pharmaceutical grade compounds of the present invention may be synthesized using a stereospecific synthesis that is scalable to a large-scale production, namely displaying adequate purity and yield.

As used herein, the term “pharmaceutical composition” refers to a compound of the present invention optionally admixed with one or more pharmaceutically acceptable carriers, diluents, or exipients. Pharmaceutical compositions preferably exhibit a degree of stability to environmental conditions so as to make them suitable for manufacturing and commercialization purposes.

As used herein, the terms “effective amount”, “therapeutic amount”, or “effective dose” refer to an amount of the compound of the present invention sufficient to elicit the desired pharmacological or therapeutic effects, thus resulting in effective prevention or treatment of a disorder. Prevention of the disorder may be manifested by delaying or preventing the progression of the disorder, as well as the onset of the symptoms associated with the disorder. Treatment of the disorder may be manifested by a decrease or elimination of symptoms, inhibition or reversal of the progression of the disorder, as well as any other contribution to the well being of the patient.

As used herein, the phrase “substantially crystalline” includes greater than 20%, or greater than 30%, and or greater than 40% (e.g. greater than any of 50, 60, 70, 80, or 90%) crystalline.

As used herein, the phrase “substantially’ or ‘sufficiently’ quality, purity or pure, includes greater than 20%, preferably greater than 30%, and more preferably greater than 40% (e.g. greater than any of 50, 60, 70, 80, or 90%) quality or purity.

The term “stability” as defined herein includes chemical stability and solid state stability, where the phrase “chemical stability” includes the potential to store salts of the invention in an isolated form, or in the form of a formulation in which it is provided in admixture with pharmaceutically acceptable carriers, diluents, excipients, or adjuvants, such as in an oral dosage form, such as a tablet, capsule, or the like, under normal storage conditions, with an insignificant degree of chemical degradation or decomposition, and the phrase “solid state stability”, includes the potential to store salts of the invention in an isolated solid form, or in the form of a solid formulation in which it is provided n admixture with pharmaceutically acceptable carriers, diluents, excipients, or adjuvants, such as in an oral dosage form, such as a tablet, capsule, or the like, under normal storage conditions, with an insignificant degree of solid state transformation, such as crystallization, recrystallization, solid slate phase transition, hydration, dehydration, solvation, or desolvation.

Examples of “normal storage conditions” include one or more of temperatures of between −80° C. and 50° C., preferably between 0° C. and 40° C. and more preferably ambient temperatures, such as 15° C. to 30° C., pressures of between 0.1 and 2 bars, preferably at atmospheric pressure, relative humidity of between 5 and 95%, preferably 10 to 60%, and exposure to 460 lux or less of UV/visible light, for prolonged periods, such as greater than or equal to six months. Under such conditions, salts of the invention may be found to be less than 5%, more preferably less than 2%, and especially less than 1%, chemically degraded or decomposed, or solid state transformed, as appropriate. The skilled person will appreciate that the above-mentioned upper and lower limits for temperature, pressure, and relative humidity represent extremes of normal storage conditions, and that certain combinations of these extremes will not be experienced during normal storage (e.g. a temperature of 50° C. and a pressure of 0.1 bar).

### I. Scalable Synthesis of 7-(3-pyridinyl)-1,7-diazaspiro[4.4]nonane

A novel method for the preparation of 7-(3-pyridinyl)-1,7-diazaspiro[4.4]nonane, a method for the separation of the compound into its component enantiomers using (−)-di-O,O′-p-toluoyl-L-tartaric acid and/or (+)-di-O,O′-p-toluoyl-D-tartaric acid, novel salt forms of 7-(3-pyridinyl)-1,7-diazaspiro[4.4]nonane, novel salt forms of (R)- and (S)-7-(3-pyridinyl)-1,7-diazaspiro[4.4]nonane, pharmaceutical compositions including the racemic and enantiomeric salt forms, methods of preparing the racemic and enantiomeric salt forms, and methods of treatment and/or prevention using the salt forms, are described in detail below.

As is well known to those of skill in the art of organic synthesis, particular synthetic steps vary in their amenability to scale-up. Reactions are found lacking in their ability to be scaled-up for a variety of reasons, including safety concerns, reagent expense, difficult work-up or purification, reaction energetics (thermodynamics or kinetics), and reaction yield. The synthesis of 7-(3-pyridinyl)-1,7-diazaspiro[4.4]nonane described herein has been used to produce kilogram quantities of material, and the component reactions have been carried out on multi-kilogram scale in high yield. The synthesis of 7-(3-pyridinyl)-1,7-diazaspiro[4.4]nonane described herein could be used in cGMP commercial scale active pharmaceutical ingredient (API) manufacture. The synthetic sequence reported herein avoids chromatographic purifications and expensive reagents.

### II. Novel Salt forms of 7-(3-pyridinyl)-1,7-diazaspiro[4.4]nonane

As can be readily appreciated, certain salt forms are more amenable to drug development than others. After screening a number of potential salt forms, the salt forms described herein were determined to have optimal properties for one or more of the synthesis, purification, tablet formation, and storage, of the R and S isomers, or racemic mixture thereof, of 7-(3-pyridinyl)-1,7-diazaspiro[4.4]nonane.

The novel salt forms of 7-(3-pyridinyl)-1,7-diazaspiro[4.4]nonane described herein include salt compositions that possess anions derived from succinic acid and oxalic acid. The stoichiometry of the salts comprising the present invention can vary. That is, the free base compound 7-(3-pyridinyl)-1,7-diazaspiro[4.4]nonane can protonate (i.e., abstract a hydrogen ion from a protic acid) at one or two sites (e.g., at the secondary amine site of the spirocycle and at the pyridine nitrogen) to give mono- or di-cationic species. Similarly, some pharmaceutically acceptable acids, such as succinic acid, are di-protic (i.e., contain two acidic hydrogens), and still others, such as phosphoric acid, are tri-protic. Thus, various ratios of base to acid, in the salts of 7-(3-pyridinyl)-1,7-diazaspiro[4.4]nonane, including 1:1, 1:2, 2:1, 3:2, 2:3, 1:3, and 3:1, are contemplated.

Also, depending upon the manner by which the salts described herein are formed, the salts can have crystal structures that occlude solvent that are present during salt formation. Thus, the salts can occur as hydrates and other solvates of varying stoichiometry of solvent relative to the 7-(3-pyridinyl)-1,7-diazaspiro[4.4]nonane salt. The scope of the present invention includes hydrated and solvated forms of 7-(3-pyridinyl)-1,7-diazaspiro[4.4]nonane or a salt thereof.

In one embodiment, the 7-(3-pyridinyl)-1,7-diazaspiro[4.4]nonane or a pharmaceutically acceptable salt thereof is substantially pure stereoisomerically. In one embodiment, the (R)-7-(3-pyridinyl)-1,7-diazaspiro[4.4]nonane or a pharmaceutically acceptable salt thereof is substantially free of (S)-7-(3-pyridinyl)-1,7-diazaspiro[4.4]nonane. In one embodiment, the (R)-7-(3-pyridinyl)-1,7-diazaspiro[4.4]nonane or a pharmaceutically acceptable salt thereof is present in an amount of about 75% by weight compared to (S)-7-(3-pyridinyl)-1,7-diazaspiro[4.4]nonane, preferably greater than 85% by weight, more preferably greater than 95% by weight, more preferably greater than 98% by weight, and most preferably 99% by weight or greater. One embodiment relates to 100% pure (R)-7-(3-pyridinyl)-1,7-diazaspiro[4.4]nonane.

The method for preparing the salt forms can vary. The preparation of 7-(3-pyridinyl)-1,7-diazaspiro[4.4]nonane salt forms involves:

(i) mixing the free base or a solution of the free base of suitably pure 7-(3-pyridinyl)-1,7-diazaspiro[4.4]nonane in a suitable solvent with any of the acids in pure form or as a solution of any of the acids in a suitable solvent,

(iia) cooling the resulting salt solution if necessary to cause precipitation, or

(iib) adding a suitable anti-solvent to cause precipitation, or

(iic) evaporating the first solvent and adding and new solvent and repeating either steps (iia) or step (iib), and

(iii) filtering and collecting the salt.

The stoichiometry, solvent mix, solute concentration, and temperature employed can vary. Representative solvents that can be used to prepare and/or recrystallize the salt forms include, without limitation, ethanol, methanol, isopropyl alcohol, acetone, ethyl acetate, and acetonitrile.

### III. Resolution of 7-(3-pyridinyl)-1,7-diazaspiro[4.4]nonane into its enantiomers

Racemic active pharmaceutical ingredient have been separated into individual isomers by classical resolution methods using single enantiomer forms of chiral organic acids. See, for example, Evans, G. R. et al. Development of Highly Efficient Resolutions of Racemic Tramadol Using Mandelic Acid in Organic Process Research & Development 2002; Vol. 6, 729-37. Synthetic intermediates have also been separated into individual stereoisomers by resolution with chiral acids and the intermediates have then been converted to the active pharmaceutical ingredients. See, for example, Taber et al., Organic Process Research and Development 8: 385-388 (2004) and U.S. Pat. No. 6,995,286 to Cipla Limited, Mumbai, India. D- and L-Di-O,O′-p-toluoyltartaric acids are among the acids that have been used in the resolution of racemic organic bases (see, for instance, Schaus et al., Synth. Comm. 20(22): 3553-3562 (1990) and Acs et al., Tetrahedron Lett. 32(49): 7325-7328 (1991)), but these acids have not previously been reported for the resolution of 7-(3-pyridinyl)-1,7-diazaspiro[4.4]nonane into its enantiomers.

The proline amide route, previously described for the separation of 7-(3-pyridinyl)-1,7-diazaspiro[4.4]nonane into its enantiomers (U.S. Pat. No. 6,956,042), involved several synthetic steps, and column chromatographic separations. The current invention includes a more efficient separation method for the enantiomers of 7-(3-pyridinyl)-1,7-diazaspiro[4.4]nonane, using the chiral acid pair D- and L-di-O,O′-p-toluoyltartaric acid. This method affords salts of high enantiomeric purity and in high yield and can be used to effectively separate the racemic compound on a large scale.

### IV. Novel salt forms of (R)- and (S)-7-(3-pyridinyl)-1,7-diazaspiro[4.4]nonane

In contrast to the behavior of the racemic 7-(3-pyridinyl)-1,7-diazaspiro[4.4]nonane, which readily formed a solid succinate salt, neither (R)— nor (S)-7-(3-pyridinyl)-1,7-diazaspiro[4.4]nonane produced a solid succinate salt. However, other pharmaceutically acceptable acids, for example benzoic acid and p-hydroxybenzoic acid, afforded solid salts, with melting points and water solubilities appropriate to further drug development as herein described, when reacted with (R)- or (S)-7-(3-pyridinyl)-1,7-diazaspiro[4.4]nonane. In addition, numerous other acids have been found to form solid salts with one or both of the enantiomers of 7-(3-pyridinyl)-1,7-diazaspiro[4.4]nonane.

The stoichiometry of the salts comprising the present invention can vary. That is, ratios of free base to acid can vary from, for example, 1:1, 1:2, 2:1, 3:2, 2:3, 1:3, and 3:1. Also, depending upon the manner by which the salts described herein are formed, the salts can have crystal structures that occlude solvents that are present during salt formation. Thus, the salts can occur as hydrates and other solvates of varying stoichiometry of solvent relative to the (R)- or (S)-7-(3-pyridinyl)-1,7-diazaspiro[4.4]nonane salt.

The method for preparing the salt forms can vary. The preparation of (R) or (S)-7-(3-pyridinyl)-1,7-diazaspiro[4.4]nonane salt forms involves:

(i) mixing the free base or a solution of the free base of suitably pure (R)- or (S)-7-(3-pyridinyl)-1,7-diazaspiro[4.4]nonane in a suitable solvent with any of the acids in pure form or as a solution of any of the acids in a suitable solvent,
(iia) cooling the resulting salt solution if necessary to cause precipitation, or
(iib) adding a suitable anti-solvent to cause precipitation, or
(iic) evaporating the first solvent and adding and new solvent and repeating either steps (iia) or step (iib), and
(iii) filtering and collecting the salt.

The stoichiometry, solvent mix, solute concentration and temperature employed can vary. Representative solvents that can be used to prepare and/or recrystallize the salt forms include, without limitation, ethanol, methanol, isopropyl alcohol, acetone, ethyl acetate, and acetonitrile.

### V. Methods of Treatment

The compounds of the present invention, which include 7-(3-pyridinyl)-1,7-diazaspiro[4.4]nonane, (R)-7-(3-pyridinyl)-1,7-diazaspiro[4.4]nonane, (S)-7-(3-pyridinyl)-1,7-diazaspiro[4,4]nonane and their pharmaceutically acceptable salts, or a pharmaceutical composition comprising said compounds can be used for the prevention or treatment of various conditions or disorders for which other types of nicotinic compounds have been proposed or are shown to be useful as therapeutics, such as CNS disorders, inflammation, inflammatory response associated with bacterial and/or viral infection, pain, metabolic syndrome, autoimmune disorders or other disorders described in further detail herein. The compounds can also be used as a diagnostic agent in receptor binding studies (in vitro and in vivo). Such therapeutic and other teachings are described, for example, in references previously listed herein, including Williams et al., Drug News Perspec. 7(4): 205 (1994), Arneric et al., CNS Drug Rev. 1(1): 1-26 (1995), Arneric et al., Exp. Opin. Invest. Drugs 5(1): 79-100 (1996), Bencherif et al., J. Pharmacol. Exp. Ther. 279: 1413 (1996), Lippiello et al., J. Pharmacol. Exp. Ther. 279: 1422 (1996), Danaj et al., J. Pharmacol. Exp. Ther. 291: 390 (1999); Chiari et al., Anesthesiology 91: 1447 (1999), Lavand'homme and Eisenbach, Anesthesiology 91: 1455 (1999), Holladay et al., J. Med. Chem. 40(28): 4169-94 (1997), Bannon et al., Science 279: 77 (1998), PCT WO 94/08992, PCT WO 96/31475, PCT WO 96/40682, and U.S. Pat. No. 5,583,140 to Bencherif et al., U.S. Pat. No. 5,597,919 to Dull et al., U.S. Pat. No. 5,604,231 to Smith et al. and U.S. Pat. No. 5,852,041 to Cosford et al.

### CNS Disorders

The compounds of the present invention, including pharmaceutically acceptable salts, or a pharmaceutical composition comprising said compounds are useful in the treatment or prevention of a variety of CNS disorders, including neurodegenerative disorders, neuropsychiatric disorders, neurologic disorders, and addictions. The compounds and their pharmaceutical compositions can be used to treat or prevent cognitive deficits and dysfunctions, age-related and otherwise; attentional disorders and dementias, including those due to infectious agents or metabolic disturbances; to provide neuroprotection; to treat convulsions and multiple cerebral infarcts; to treat mood disorders, compulsions and addictive behaviors; to provide analgesia; to control inflammation, such as mediated by cytokines and nuclearfactor kappa B; to treat inflammatory disorders; to provide pain relief; and to treat infections, as anti-infectious agents for treating bacterial, fungal, and viral infections. Among the disorders, diseases and conditions that the compounds and pharmaceutical compositions of the present invention can be used to treat or prevent are: age-associated memory impairment (AAMI), mild cognitive impairment (MCI), age-related cognitive decline (ARCD), pre-senile dementia, early onset Alzheimer's disease, senile dementia, dementia of the Alzheimer's type, Alzheimer's disease, cognitive impairment no dementia (CIND), Lewy body dementia, HIV-dementia, AIDS dementia complex, vascular dementia, Down syndrome, head trauma, traumatic brain injury (TBI), dementia pugilistica, Creutzfeld-Jacob Disease and prion diseases, stroke, ischemia, attention deficit disorder, attention deficit hyperactivity disorder, dyslexia, schizophrenia, schizophreniform disorder, schizoaffective disorder, cognitive dysfunction in schizophrenia, cognitive deficits in schizophrenia, Parkinsonism including Parkinson's disease, postencephalitic parkinsonism, parkinsonism-dementia of Gaum, frontotemporal dementia Parkinson's Type (FTDP), Pick's disease, Niemann-Pick's Disease, Huntington's Disease, Huntington's chorea, tardive dyskinesia, hyperkinesia, progressive supranuclear palsy, progressive supranuclear paresis, restless leg syndrome, Creutzfeld-Jakob disease, multiple sclerosis, amyotrophic lateral sclerosis (ALS), motor neuron diseases (MND), multiple system atrophy (MSA), corticobasal degeneration, Guillain-Barré Syndrome (GBS), and chronic inflammatory demyelinating polyneuropathy (CIDP), epilepsy, autosomal dominant nocturnal frontal lobe epilepsy, mania, anxiety, depression, premenstrual dysphoria, panic disorders, bulimia, anorexia, binge eating, compulsive eating, narcolepsy, excessive daytime sleepiness, bipolar disorders, generalized anxiety disorder, major depressive disorder, obsessive compulsive disorder, somatoform disorders, hypochondriasis, dysthymia, seasonal affective disorder, conversion disorder, malingering, Munchausen Syndrome, rage outbursts, oppositional defiant disorder, Tourette's syndrome, autism, drug and alcohol addiction, tobacco addiction, obesity, cachexia, psoriasis, lupus, acute cholangitis, aphthous stomatitis, ulcers, asthma, ulcerative colitis, inflammatory bowel disease, Crohn's disease, spastic dystonia, diarrhea, constipation, pouchitis, viral pneumonitis, arthritis, including, rheumatoid arthritis and osteoarthritis, endotoxaemia, sepsis, atherosclerosis, idiopathic pulmonary fibrosis, acute pain, chronic pain, neuropathies, urinary incontinence, diabetes and neoplasias.

Cognitive impairments or dysfunctions may be associated with psychiatric disorders or conditions, such as schizophrenia and other psychotic disorders, including but not limited to psychotic disorder, schizophreniform disorder, schizoaffective disorder, delusional disorder, brief psychotic disorder, shared psychotic disorder, and psychotic disorders due to a general medical conditions, dementias and other cognitive disorders, including but not limited to mild cognitive impairment, pre-senile dementia, Alzheimer's disease, senile dementia, dementia of the Alzheimer's type, age-related memory impairment, Lewy body dementia, vascular dementia, AIDS dementia complex, dyslexia, Parkinsonism including Parkinson's disease, cognitive impairment and dementia of Parkinson's Disease, cognitive impairment of multiple sclerosis, cognitive impairment caused by traumatic brain injury, dementias due to other general medical conditions, anxiety disorders, including but not limited to panic disorder without agoraphobia, panic disorder with agoraphobia, agoraphobia without history of panic disorder, specific phobia, social phobia, obsessive-compulsive disorder, post-traumatic stress disorder, acute stress disorder, generalized anxiety disorder and generalized anxiety disorder due to a general medical condition, mood disorders, including but not limited to major depressive disorder, dysthymic disorder, bipolar depression, bipolar mania, bipolar I disorder, depression associated with manic, depressive or mixed episodes, bipolar II disorder, cyclothymic disorder, and mood disorders due to general medical conditions, sleep disorders, including but not limited to dyssomnia disorders, primary insomnia, primary hypersomnia, narcolepsy, parasomnia disorders, nightmare disorder, sleep terror disorder and sleepwalking disorder, mental retardation, learning disorders, motor skills disorders, communication disorders, pervasive developmental disorders, attention-deficit and disruptive behavior disorders, attention deficit disorder, attention deficit hyperactivity disorder, feeding and eating disorders of infancy, childhood, or adults, tic disorders, elimination disorders, substance-related disorders, including but not limited to substance dependence, substance abuse, substance intoxication, substance withdrawal, alcohol-related disorders, amphetamine or amphetamine-like-related disorders, caffeine-related disorders, cannabis-related disorders, cocaine-related disorders, hallucinogen-related disorders, inhalant-related disorders, nicotine-related disorders, opioid-related disorders, phencyclidine or phencyclidine-like-related disorders, and sedative-, hypnotic- or anxiolytic-related disorders, personality disorders, including but not limited to obsessive-compulsive personality disorder and impulse-control disorders.

Cognitive performance may be assessed with a validated cognitive scale, such as, for example, the cognitive subscale of the Alzheimer's Disease Assessment Scale (ADAS-cog). One measure of the effectiveness of the compounds of the present invention in improving cognition may include measuring a patients degree of change according to such a scale.

The above conditions and disorders are discussed in further detail, for example, in the American Psychiatric Association: Diagnostic and Statistical Manual of Mental Disorders, Fourth Edition, Text Revision, Washington, D.C., American Psychiatric Association, 2000. This Manual may also be referred to for greater detail on the symptoms and diagnostic features associated with substance use, abuse, and dependence.

One embodiment relates to treating CNS disorders in a subject in need thereof comprising administering to said subject 7-(3-pyridinyl)-1,7-diazaspiro[4.4]nonane (R)-7-(3-pyridinyl)-1,7-diazaspiro[4.4]nonane or (S)-7-(3-pyridinyl)-1,7-diazaspiro[4.4]nonane, or a pharmaceutically acceptable salt thereof, or a pharmaceutical composition comprising said compounds.

In another embodiment the CNS disorders are selected from depression, anxiety, bipolar disorders, mania, premenstrual dysphoria, panic disorders, bulimia, anorexia, generalized anxiety disorder, seasonal affective disorder, major depressive disorder, obsessive compulsive disorder, rage outbursts, oppositional defiant disorder, Tourette's syndrome, autism, drug and alcohol addiction, tobacco addiction, compulsive eating and obesity.

### Inflammation

The nervous system, primarily through the vagus nerve, is known to regulate the magnitude of the innate immune response by inhibiting the release of macrophage tumor necrosis factor (TNF). This physiological mechanism is known as the “cholinergic anti-inflammatory pathway” (see, for example, Tracey, “The inflammatory reflex,” Nature 420: 853-9 (2002)). Excessive inflammation and tumor necrosis factor synthesis cause morbidity and even mortality in a variety of diseases. These diseases include, but are not limited to, endotoxemia, rheumatoid arthritis, osteoarthritis, psoriasis, asthma, atherosclerosis, idiopathic pulmonary fibrosis, and inflammatory bowel disease.

Inflammatory conditions that can be treated or prevented by administering the compounds described herein include, but are not limited to, chronic and acute inflammation, psoriasis, endotoxemia, gout, acute pseudogout, acute gouty arthritis, arthritis, rheumatoid arthritis, osteoarthritis, allograft rejection, chronic transplant rejection, asthma, atherosclerosis, mononuclear-phagocyte dependent lung injury, idiopathic pulmonary fibrosis, atopic dermatitis, chronic obstructive pulmonary disease, adult respiratory distress syndrome, acute chest syndrome in sickle cell disease, inflammatory bowel disease, Crohn's disease, ulcerative colitis, acute cholangitis, aphteous stomatitis, pouchitis, glomerulonephritis, lupus nephritis, thrombosis, and graft vs. host reaction.

Inflammatory Response Associated with Bacterial and/or Viral Infection

Many bacterial and/or viral infections are associated with side effects brought on by the formation of toxins, and the body's natural response to the bacteria or virus and/or the toxins. As discussed above, the body's response to infection often involves generating a significant amount of TNF and/or other cytokines. The over-expression of these cytokines can result in significant injury, such as septic shock (when the bacteria is sepsis), endotoxic shock, urosepsis and toxic shock syndrome.

Cytokine expression is mediated by NNRs, and can be inhibited by administering agonists or partial agonists of these receptors. Those compounds described herein that are agonists or partial agonists of these receptors can therefore be used to minimize the inflammatory response associated with bacterial infection, as well as viral and fungal infections. Examples of such bacterial infections include anthrax, botulism, and sepsis. Some of these compounds may also have antimicrobial properties.

The compounds of the present invention may also be used as adjunct therapy in combination with existing therapies to manage bacterial, viral and fungal infections, such as antibiotics, antivirals and antifungals. Antitoxins may also be used to bind to toxins produced by the infectious agents and allow the bound toxins to pass through the body without generating an inflammatory response. Examples of antitoxins are disclosed, for example, in U.S. Pat. No. 6,310,043 to Bundle et al. Other agents effective against bacterial and other toxins can be effective and their therapeutic effect can be complemented by co-administration with the compounds described herein.

### Pain

The compounds can be administered to treat and/or prevent pain, including acute, neurologic, inflammatory, neuropathic and chronic pain. The analgesic activity of compounds described herein can be demonstrated in models of persistent inflammatory pain and of neuropathic pain, performed as described in U.S. Published Patent Application No. 20010056084 A1 (Allgeier et al.) (e.g., mechanical hyperalgesia ii the complete Freund's adjuvant rat model of inflammatory pain and mechanical hyperalgesia in the mouse partial sciatic nerve ligation model of neuropathic pain).

The analgesic effect is suitable for treating pain of various genesis or etiology, in particular in treating inflammatory pain and associated hyperalgesia, neuropathic pain and associated hyperalgesia, chronic pain (e.g., severe chronic pain, post-operative pain and pain associated with various conditions including cancer, angina, renal or biliary colic, menstruation, migraine and gout). Inflammatory pain may be of diverse genesis, including arthritis and rheumatoid disease, teno-synovitis and vasculitis. Neuropathic pain includes trigeminal or herpetic neuralgia, diabetic neuropathy pain, causalgia, low back pain and deafferentation syndromes such as brachial plexus avulsion.

### Other Disorders

In addition to treating CNS disorders, inflammation, and pain, the compounds of the present invention may be also used to prevent or treat certain other conditions, diseases, and disorders in which NNRs play a role. Examples include autoimmune disorders such as Lupus, disorders associated with cytokine release, cachexia secondary to infection (e.g., as occurs in AIDS, AIDS related complex and neoplasia), obesity, pemphitis, urinary incontinence, retinal diseases, infenctious diseases, myasthenia, Eaton-Lambert syndrome, hypertension, osteoporosis, vasoconstriction, vasodilatation, cardiac arrhythmias, type I diabetes, bulimia, anorexia as well as those indications set forth in published PCT application WO 98/25619. The compounds of this invention may also be administered to treat convulsions such as those that are symptomatic of epilepsy, and to treat conditions such as syphillis and Creutzfeld-Jakob disease.

### Diagnostic Uses

The compounds may be used in diagnostic compositions, such as probes, particularly when they are modified to include appropriate labels. The probes may be used, for example, to determine the relative number and/or function of specific receptors, particularly the α4β2 receptor subtype. For this purpose the compounds of the present invention most preferably are labeled with a radioactive isotopic moiety such as ¹¹C, ¹⁸F, ⁷⁶Br, ¹²³I or ¹²⁵I.

The administered compounds can be detected using known detection methods appropriate for the label used. Examples of detection methods include position emission topography (PET) and single-photon emission computed tomography (SPECT). The radiolabels described above are useful in PET (e.g., ¹¹C, ¹⁸F or ⁷⁶Br) and SPECT (e.g., ¹²³I) imaging, with half-lives of about 20.4 min for ¹¹C, about 109 min for ¹⁸F, about 13 h for ¹²³I, and about 16 h for ⁷⁶Br. A high specific activity is desired to visualize the selected receptor subtypes at non-saturating concentrations. The administered doses typically are below the toxic range and provide high contrast images. The compounds are expected to be capable of administration in non-toxic levels. Determination of dose is carried out in a manner known to one skilled in the art of radiolabel imaging. See, for example, U.S. Pat. No. 5,969,144 to London et al.

The compounds may be administered using known techniques. See, for example, U.S. Pat. No. 5,969,144 to London et al. The compounds may be administered in compositions that incorporate other ingredient, such as those types of ingredients that are useful in formulating a diagnostic composition. Compounds useful in accordance with carrying out the present invention most preferably are employed in forms of high purity. See, U.S. Pat. No. 5,853,696 to Elmalch et al.

After the compounds are administered to a subject (e.g., a human subject), the presence of that compound within the subject can be imaged and quantified by appropriate techniques in order to indicate the presence, quantity, and functionality of selected NNR subtypes. In addition to humans, the compounds may also be administered to animals, such as mice, rats, horses, dogs, and monkeys. SPECT and PET imaging can be carried out using any appropriate technique and apparatus. See Villemagne et al., In: Arneric et al. (Eds.) Neuronal Nicotinic Receptors: Pharmacology and Therapeutic Opportunities, 235-250 (1998) and U.S. Pat. No. 5,853,696 to Elmalch et al.

The radiolabeled compounds bind with high affinity to selective NNR subtypes (e.g., α4β2) and preferably exhibit negligible non-specific binding to other nicotinic cholinergic receptor subtypes (e.g., those receptor subtypes associated with muscle and ganglia). As such, the compounds can be used as agent for noninvasive imaging of nicotinic cholinergic receptor subtypes within the body of a subject, particularly within the brain for diagnosis associated with a variety of CNS diseases and disorders.

In one aspect, the diagnostic compositions may be used in a method to diagnose disease in a subject, such as a human patient. The method involves administering to that patient a detectably labeled compound as described herein, and detecting the binding of that compound to selected NNR subtypes (e.g., α4β2 receptor subtypes). Those skilled in the art of using diagnostic tools, such as PET and SPECT, can use the radiolabeled compounds described herein to diagnose a wide variety of conditions and disorders, including conditions and disorders associated with dysfunction of the central and autonomic nervous systems. Such disorders include a wide variety of CNS diseases and disorders, including Alzheimer's disease, Parkinson's disease, and schizophrenia. These and other representative diseases and disorders that may be treated include those that are set forth in U.S. Pat. No. 5,952,339 to Bencherif et al.

In another aspect, the diagnostic compositions can be used in a method to monitor selective nicotinic receptor subtypes of a subject, such as a human patient. The method involves administering a detectably labeled compound as described herein to that patient and detecting the binding of that compound to selected nicotinic receptor subtypes namely, the α4β2 receptor subtypes.

### Receptor Binding

The compounds of this invention may be used as reference ligands in binding assays for compounds which bind to NNR subtypes, particularly the α4β2 receptor subtypes. For this purpose the compounds of this invention are preferably labeled with a radioactive isotopic moiety such as ³H, or ¹⁴C. Examples of such binding assays are described in detail below.

### VI. Pharmaceutical Compositions

Although it is possible to administer the compounds of the present invention in the form of a bulk active chemical, it is preferred to administer the compounds in the form of a pharmaceutical composition or formulation. Thus, in one aspect the present invention relates to pharmaceutical compositions comprising the compounds of the present invention and one or more pharmaceutically acceptable carrier, diluent, or excipient. Another aspect of the invention provides a process for the preparation of a pharmaceutical composition including admixing the compounds of the present invention with one or more pharmaceutically acceptable carrier, diluent, or excipient.

The manner in which the compounds of the present invention are administered can vary. The compounds of the present invention are preferably administered orally. Preferred pharmaceutical compositions for oral administration include tablets, capsules, caplets, syrups, solutions, and suspensions. The pharmaceutical compositions of the present invention may be provided in modified release dosage forms such as time-release tablet and capsule formulations.

The pharmaceutical compositions may also be administered via injection, namely, intravenously, intramuscularly, subcutaneously, intraperitoneally, intraarterially, intrathecally, and intracerebroventricularly. Intravenous administration is a preferred method of injection. Suitable carriers for injection are well known to those of skill in the art and include 5% dextrose solutions, saline, and phosphate buffered saline.

The compositions may also be administered using other means, for example, rectal administration. Compositions useful for rectal administration, such as suppositories, are well known to those of skill in the art. The compounds may also be administered by inhalation, for example, in the form of an aerosol; topically, such as, in lotion form; transdermally, such as, using a transdermal patch (for example, by using technology that is commercially available from Novartis and Alza Corporation), by powder injection, or by buccal, sublingual, or intranasal absorption.

Pharmaceutical compositions may be formulated in unit dose form, or in multiple or subunit doses forms.

The administration of the pharmaceutical compositions described herein can be intermittent, or at a gradual, continuous, constant or controlled rata. The pharmaceutical compositions may be administered to a warm-blooded animal, for example, a mammal such as a mouse, rat, cat, rabbit, horses, dog, pig, cow, or monkey; but advantageously is administered to a human being. The compounds of the present invention may be used in the treatment of a variety of disorders and conditions and, as such, may be used in combination with a variety of other therapeutic agents useful in the treatment or prophylaxis of those disorders. Thus, one embodiment of the present invention relates to the administration of the compounds of the present invention in combination with other therapeutic agents. For example, the compounds of the present invention may be used in combination with other NNR ligands (such as varenicline), antioxidants (such as free radical scavenging agents), antibacterial agents (such as penicillin antibiotics), antiviral agents (such as nucleoside analogs, like zidovudine and acyclovir), anticoagulants (such as warfarin), anti-inflammatory agents (such as NSAIDs), anti-pyretics, analgesics, anesthetics (such as used in surgery), acetylcholinesterase inhibitors (such as donepezil and galantamine), antipsychotics (such as haloperidol, clozapine, olanzapine, and quetiapine), immuno-suppressants (such as cyclosporin and methotrexate), neuroprotective agents, steroids (such as steroid hormones), corticosteroids (such as dexamethasone, predisone, and hydrocortisone), vitamins, minerals, nutraceuticals, anti-depressants (such as imipramine, fluoxetine, paroxetine, escitalopram, sertraline, venlafaxine, and duloxetine), anxiolytics (such as alprazolam and buspirone), anticonvulsants (such as phenyloin and gabapentin), vasodilators (such as prazosin and sildenafil), mood stabilizers (such as valproate and aripiprazole), anti-cancer drugs (such as anti-proliferatives), antihypertensive agents (such as atenolol, clonidine, amlopidine, verapamil, and olmesartan), laxatives, stool softeners, diuretics (such as furosemide), anti-spasmotics (such as dicyclomine), anti-dyskinetic agents, and anti-ulcer medications (such as esomeprazole). Such a combination of therapeutic agents may be administered together or separately and, when administered separately, administration may occur simultaneously or sequentially, in any order. The amounts of the compounds or agents and the relative timings of administration will be selected in order to achieve the desired therapeutic effect. The administration in combination of compounds of the present invention with other therapeutic agents may be in combination by administration concomitantly in: (1) a unitary pharmaceutical composition including both compounds; or (2) separate pharmaceutical compositions each including one of the compounds. Alternatively, the combination may be administered separately in a sequential manner wherein one treatment agent is administered first and the other second. Such sequential administration may be close in time or remote in time.

Another aspect of the present invention relates to combination therapy comprising administering to the subject a therapeutically or prophylactically effective amount of the compounds of the present invention and one or more other therapeutic agents including chemotherapeutics, radiation therapeutic agents, gene therapeutic agents, or agents used in immunotherapy.

### VII. Examples

The following synthetic and analytical examples are provided to illustrate the present invention, and should not be construed as limiting thereof. In these examples, all parts and percentages are by weight, unless otherwise noted. Reaction yields are reported in mole percentages.

### Example 1

### Determination of Binding to Receptor Sites

Binding and function of the compounds to relevant receptor sites was determined in accordance with the techniques described in PCT WO 2008/057938. Inhibition constants (Kᵢ values), reported in nM, were calculated from the IC₅₀ values using the method of Cheng et al., Biochem. Pharmacol. 22: 3099 (1973). Low inhibition constants indicate that the compounds of the present invention exhibit high affinity binding to NNRs. 7-(3-Pyridinyl)-1,7-diazaspiro[4.4]nonane, (R)-7-(3-pyridinyl)-1,7-diazaspiro[4.4]nonane, and (S)-7-(3-pyridinyl)-1,7-diazaspiro[4.4]nonane all exhibit very high affinity for α4β2 NNRs, having Kᵢ values of less than 100 nM. The compounds of the present invention are selective for α4β2 NNRs over α7, human muscle and human ganglion subtypes, at which they exhibit little if any binding or function (see Table 1). Thus, the compounds of the present invention are selective modulators of the α4β2 NNR subtype.

However, the enantiomers of 7-(3-pyridinyl)-1,7-diazaspiro[4.4]nonane differ in their ability to activate human α4β2 NNRs. As seen in Table 1, (R)-7-(3-pyridinyl)-1,7-diazaspiro[4.4]nonane distinguishes from the racemate and the corresponding S enantiomer in that it is robustly antagonistic of the receptor (Eₘₐₓ=9% of the nicotine response; EC₅₀=84 μM). Thus, the R enantiomer should be particularly effective at counteracting hypercholinergic tone and thereby treating conditions and disorders that are associated with hypercholinergic tone, such as depression and anxiety.

TABLE 1

| | | | | Human | | | |
|---|---|---|---|---|---|---|---|
| | Rat | | | α4β2 | Human | | |
| | α4β2 | Human | Rat α7 | Emax (% | α4β2 | Human | Human |
| | Ki | α4β2 Ki | Ki | of nic | EC50 | Ganglion | Muscle |
| Compound | (nM) | (nM) | (nM) | resp) | (nM) | SP (100 μM) | SP (100 μM) |
| Racemate | 68 | 34 | >10k | 43 | 9100 | 15 | 22 |
| S enantiomer | 82 | 30 | >10k | 87 | 3900 | 18 | <1 |
| R enantiomer | 74 | 40 | 5400 | 9 | 84000 | 3 | 6 |

Function (Eₘₐₓ and EC₅₀) at human α4β2 NNRs was determined as follows: The recombinant cell line SH-EP1/human a4b2 grown in culture, was loaded with FLIPR Calcium 4 Assay Reagent (Molecular Devices) for 1 hour at either 29° C. After the loading period, plates were equilibrated to room temperature and the cells exposed to the test article (0.01 to 100 mM) or nicotine or buffer alone on a FLIPR (Molecular Devices). Fluorescence (at 485 nm) was monitored throughout the experiment. The test article change in fluorescence was compared to both a positive control (10 μM nicotine) and a negative control (buffer alone) to determine the percent response relative to that of nicotine.

To reiterate for ease of reference, (R)-7-(3-pyridinyl)-1,7-diazaspiro[4.4]nonane is referred to as Compound A. Compound B is (S)-7-(3-pyridinyl)-1,7-diazaspiro[4.4]nonane. Compound C is a racemic mixture of (R)-7-(3-pyridinyl)-1,7-diazaspiro[4.4]nonane and (S)-7-(3-pyridinyl)-1,7-diazaspiro[4.4]nonane.

Both Compound A, (R)-7-(3-pyridinyl)-1,7-diazaspiro[4.4]nonane, and Compound B, (S)-7-(3-pyridinyl)-1,7-diazaspiro[4.4]nonane, each as their hydrochloride salts, were screened, at 10 μM concentration, against a standard set of receptors. Compound B, (S)-7-(3-pyridinyl)-1,7-diazaspiro[4.4]nonane, exhibited binding at histamine H3 (58% inhibition), muscarinic M1 (53% inhibition), non-selective central muscarinic (84% inhibition), non-selective peripheral muscarinic (84% inhibition), nicotinic (99% inhibition) and non-elective sigma (56% inhibition) receptors. In contrast, Compound A, (R)-7-(3-pyridinyl)-1,7-diazaspiro[4.4]nonane, exhibited binding only at histamine H1 (66% inhibition) and nicotinic (99% inhibition) receptors. Thus, the two stereoisomers differentiate from one another in terms of their non-nicotinic receptor binding characteristics. This differentiation is believed to translate into a differentiation between the ability of each of (R)-7-(3-pyridinyl)-1,7-diazaspiro[4.4]nonane and (S)-7-(3-pyridinyl)-1,7-diazaspiro[4.4]nonane to treat various disease states, including, for example, the ability of Compound A to enhance desensitization of α4β2 NNRs. As hereinbelow described in further detail, Compound A has demonstrated efficacy in multiple validated animal models of depression and anxiety, for which Compound B failed to demonstrate activity.

### Example 1A

### Anxiety Model—Elevated Plus Maze (EPM)

Experimental Procedure

The method, which detects anxiolytic activity, follows that described by Handley S. L. and Mithani S., Effects of alpha-adrenoceptor agonists and antagonists in a maze-exploration model of fear-motivated behaviour, Naunyn-Schmied. Arch. Pharmacol., 327, 1-5, 1984.

Rodents avoid open spaces (the open arms of an elevated plus-maze). Anxiolytics increase exploratory activity in the open arms. The maze consisted of 4 arms of equal length and width arranged in the form of a plus sign (+). Two opposite arms were enclosed by walls (closed arms). The two other arms had no walls (open arms). The maze was raised above the floor. A rat was placed in the centre of the plus-maze and left to explore for 5 minutes. The number of entries into the open and closed arms and the time spent in the open arms were recorded.

Ten (10) rats were studied per group. The test was performed blind. Compound A was evaluated at 0.02, 0.06, and 0.21 mg/kg, administered i.p. 30 minutes before the test, and compared with a vehicle control group. Clobazam, administered under the same experimental conditions, was used as reference substance. The experiment therefore included 6 groups.

Statistical Analysis

Data were analyzed by comparing the treated groups with the control group using unpaired tests.

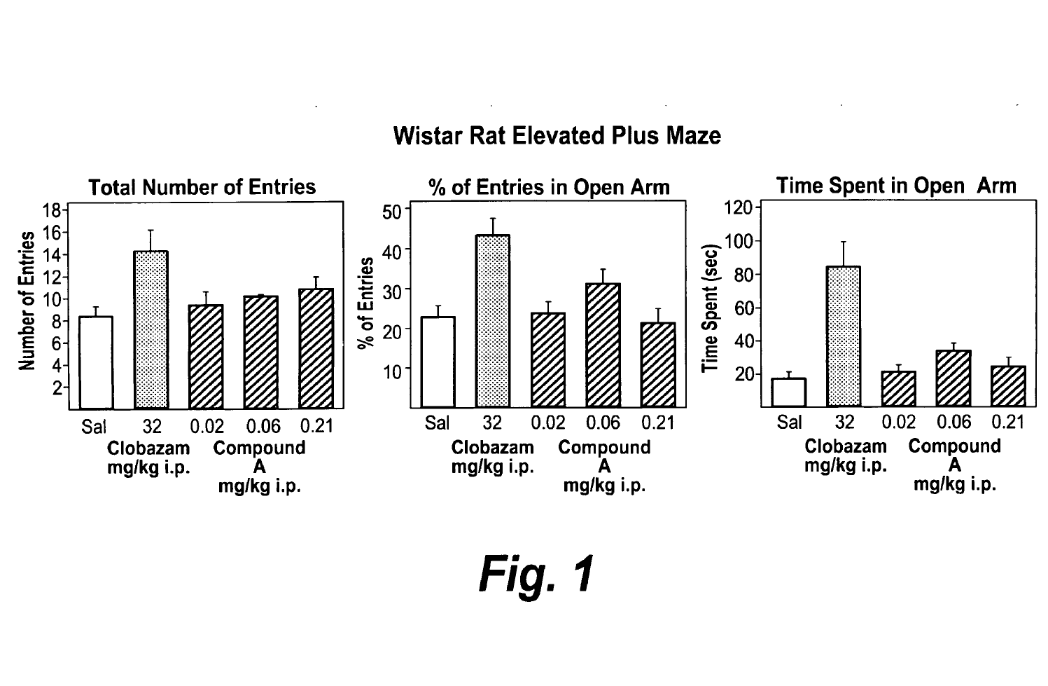

As shown in FIG. 1, Compound A exhibit anxiolytic-like activity in the EPM.

### Example 1B

### Depression Model—Tail Suspension (TS)

Tail Suspension

On the day of the test, A/J mice were brought to acclimate to the testing room for one hour. Eight animals were tested in each run. Following pretreatment with vehicle, desipramine (20 mg/kg) or Compound A, p-hydroxybenzoate (0.1, 0.3, and 1 mg/kg), a piece of transparent (Scotch) tape was attached to the tail of each mouse from about mid-tail with approximately 2 cm of tape past the end of the tail. The mice were then placed in the tail suspension chambers (white polyvinylchloride cubicles measuring 33×33×31.75 cm; Med Associates Inc., St Albans, Vt.). The mice were suspended from the hook of the TS force transducer via the tail tape. The force transducer transmitted the movements of the mouse to a recording device connected to a computer. Immobility time, struggle time, and intensity were automatically recorded for each min during the 10 min test period. Upon completion of the TS test, the mice were returned to their home cage and then to the animal colony. The TS chambers were cleaned between sessions. Data were analyzed by repeated measures and one-way analysis of variance (ANOVA) followed by Fisher PLSD post-hoc comparisons. An effect was considered significant if p<0.05.

Immobility Time

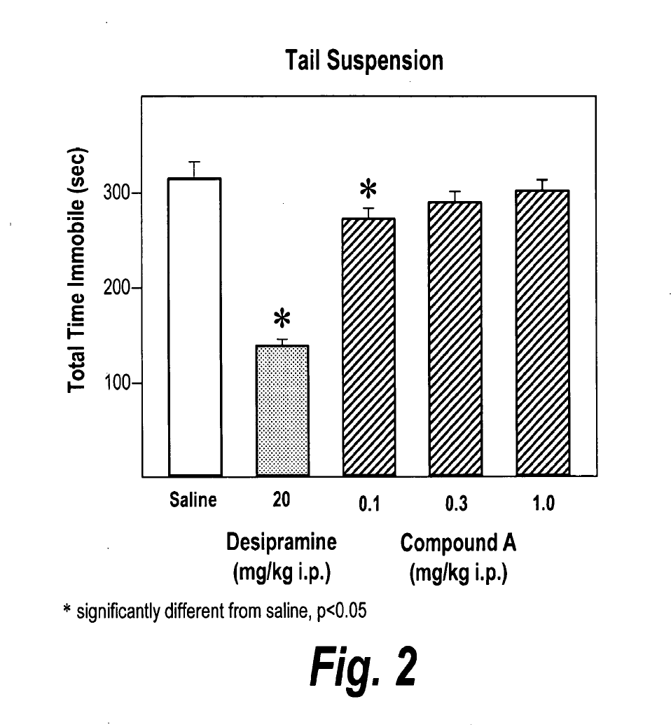

One-way ANOVA of total time immobile indicated a significant treatment effect. Post-hoc comparisons found that desipramine and Compound A (0.1 mg/kg) decreased total time immobile compared to saline. As shown in FIG. 2, Compound A effectively immobilizes the subjects' tail over the 10 minute test period. The data represent the mean±SEM.

Struggle Intensity

One-way ANOVA of total struggle intensity indicated a significant treatment effect. Post-hoc comparisons found that desipramine and Compound A (0.1 mg/kg) increased struggle intensity compared to saline.

Struggle Frequency

One-way ANOVA of total struggle frequency indicated no significant treatment effect.

### Example 1C

### Plasma Pharmacokinetic Data

Similar to the above-noted differentiation observed, likewise the compounds exhibit differential pharmacokinetic profiles. As is known, pharmacokinetic parameters, such as bioavailabiity, can be calculated from the plasma concentration vs. time profile of any particular test compound.

A single, rising dose (SRD) study was performed with Compound C (the racemate). Plasma samples were analyzed for the content of the two enantiomers, Compound A (substantially pure R), Compound B (substantially pure S). Three dose groups (Compound C) were analyzed, 50, 100, and 400 mg, and four subjects were randomly selected from the 6 subjects tested to receive active treatment in each cohort examined in the SRD study.

A difference in the terminal elimination half-life was observed, where the terminal elimination half-life estimated for Compound A was approximately 6 hours longer than the terminal elimination half-life estimated for Compound B. The data is summarized below in Table 2.

TABLE 2
Mean (SD) Terminal Elimination Half-life Data Summary

| | | 50 mg (n = 4) | 100 mg (n = 4) | 400 mg (n = 4) |
|---|---|---|---|---|
| Half- | Compound C | 24.8 (3.14) | 21.1 (1.69) | 18.5 (2.06) |
| life | Compound B | 21.0 (1.53) | 17.8 (1.87) | 16.6 (1.58) |
| (hr) | Compound A | 27.5 (2.78) | 23.7 (2.52) | 22.4 (1.89) |

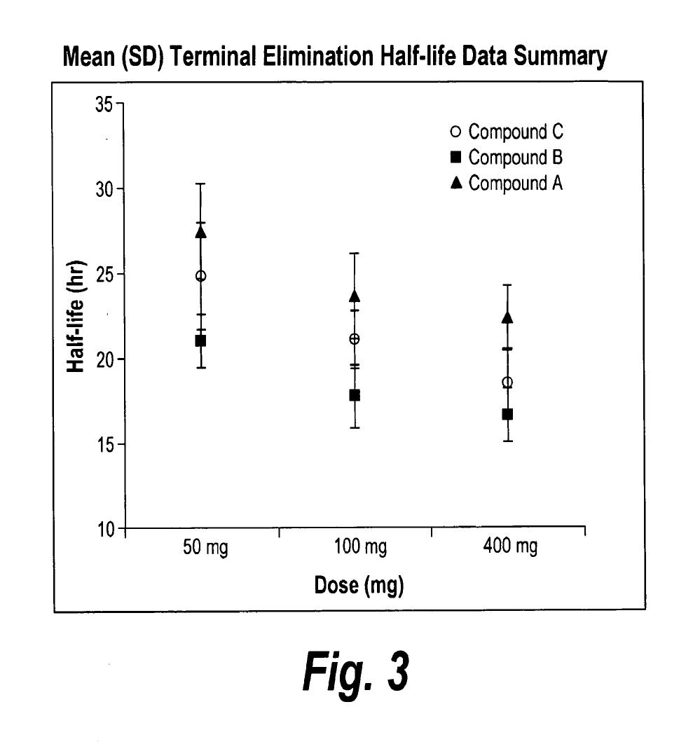

Table 3 presents an overall summary of the PK analysis. A graphical representation is presented in FIG. 3.

Regarding exposure, Cₘₐₓ and AUC_{inf}, each enantiomer, Compound A and Compound B, represented approximately half of the total exposure as compared to oral administration of the racemate, Compound C. The observed Tₘₐₓ of the enantiomers is similar.

TABLE 3
Overall Summary of PK Analysis

| | | | |
|---|---|---|---|
| PK Parameter | 50 mg (n = 4) | 100 mg (n = 4) | 400 mg (n = 4) |

Compound C: Mean (SD) PK Parameter Summary

| | | | | | | |
|---|---|---|---|---|---|---|
| Cₘₐₓ (ng/mL) | 21.8 | (5.44) | 44.7 | (7.05) | 295 | (53.3) |
| Tₘₐₓ (hr) | 4.38 | (1.89) | 31.13 | (2.02) | 2.00 | (0.707) |
| AUC_{inf} (ng*hr/mL) | 473 | (93.4) | 741 | (147) | 3676 | (915) |
| t_{1/2 (hr)} | 24.8 | (3.14) | 21.1 | (1.69) | 18.5 | (2.06) |

Compound B: Mean (SD) PK Parameter Summary

| | | | | | | |
|---|---|---|---|---|---|---|
| Cₘₐₓ (ng/mL) | 12.1 | (4.09) | 24.6 | (3.59) | 158 | (32.1) |
| Tₘₐₓ (hr) | 3.13 | (0.629) | 2.75 | (0.957) | 2.00 | (0.707) |
| AUC_{inf} (ng*hr/mL) | 237 | (63.9) | 364 | (71.5) | 2134 | (530) |
| t_{1/2 (hr)} | 21.0 | (1.53) | 17.8 | (1.87) | 16.6 | (1.58) |

Compound A: Mean (SD) Parameter Summary

| | | | | | | |
|---|---|---|---|---|---|---|
| Cₘₐₓ (ng/mL) | 10.3 | (3.27) | 22.7 | (4.23) | 163 | (29.1) |
| Tₘₐₓ (hr) | 4.38 | (1.89) | 3.25 | (1.89) | 2.00 | (0.707) |
| AUC_{inf} (ng*hr/mL) | 256 | (27.8) | 451 | (61.3) | 2418 | (408) |
| t_{1/2 (hr)} | 27.5 | (2.78) | 23.7 | (2.52) | 22.4 | (1.89) |

### Example 1D

### Side Effect Profile

Compound A is believed to exhibit a more favorable side effect profile compared to either the racemate, Compound C, or the other stereoisomer, Compound B.

For example, preclinically, Compound C induced seizures following acute doses:

| | |
|---|---|
| 1/5 female mice | Acute oral dose of 400 mg/kg Compound C |
| 1/5 male rats | Acute oral dose of 800 mg/kg Compound C |
| 1/5 female rats | Acute oral dose of 800 mg/kg Compound C |
| 1/5 female rats | Acute oral dose of 200 mg/kg Compound C |
| 1/5 male rats | Acute IV dose of 100 mg/kg Compound C |
| 4/5 female rats | Acute IV dose of 100 mg/kg Compound C |

Likewise, Compound B induced convulsion in 1/6 male rats following an acute oral dose of 300 mg/kg.

Compound A, however, had no effect on seizure induction under the same conditions. In fact, the effect of Compound A was not statistically different from the vehicle control.

### Example 2

### Scalable Synthesis of 7-(3-pyridinyl)-1,7-diazaspiro[4.4]nonane

### Methyl 1-benzoylpyrrolidine-2-carboxylate

A 22 L four neck round bottom flask, fitted with an overhead polytetrafluoroethylene (PTFE) paddle stirrer, addition funnel, nitrogen inlet, and thermometer probe, was charged with L-proline (200 g, 1.74 mol), potassium carbonate (600 g, 4.34 mol), water (2 L), and tetrahydrofuran (THF) (200 mL). This mixture was stirred under nitrogen and cooled in an ice bath as benzoyl chloride (256 g, 1.82 mol) was added via the addition funnel over 2.5 h while maintaining the internal temperature at or below 5° C. during the addition. When HPLC analysis indicated that the reaction was complete, the ice bath was removed and the mixture was allowed to warm to ambient temperature. The THF was removed by rotary evaporation and dichloromethane (2 L) was added. To this cooled (15° C.), stirred mixture was added 6 M hydrochloric acid (1.2 L) to adjust the aqueous layer to pH 1. The dichloromethane layer was removed, and the aqueous layer was extracted with dichloromethane (3×800 mL). The combined dichloromethane layers were washed with saturated aqueous sodium chloride and concentrated by rotary evaporation to give 400 g of 1-benzoylpyrrolidine-2-carboxylic acid as a white solid (mp=157-159.5° C.).

The 1-benzoylpyrrolidine-2-carboxylic acid was dissolved in methanol (1925 mL) and cooled to ˜10° C. (ice water bath) in a 5 L three-neck flask fitted with a nitrogen inlet, addition funnel, and thermometer probe. Under a nitrogen atmosphere, thionyl chloride (270 g, 2.27 mol) was added drop-wise over a 2 h period to the magnetically stirred solution while maintaining the temperature of the reaction mixture below 20° C. The mixture was stirred overnight at ambient temperature and then concentrated by rotary evaporation. The residue, which crystallized upon cooling, was dissolved in toluene (350 mL) and again concentrated. The resulting solid was dissolved in dichloromethane (800 mL) and stirred with 1 M sodium bicarbonate (400 mL) to remove un-reacted 1-benzoylpyrrolidine-2-carboxylic acid. The separated dichloromethane layer was washed with water (400 mL) and concentrated by rotary evaporation. The resulting solid was stirred in heptane (1.2 L) and collected by suction filtration. The solid was vacuum dried (50° C. for 5 h), to give 334 g of methyl 1-benzoylpyrrolidine-2-carboxylate as a white solid (82.4% yield, mp=88.5-90° C.).

### Methyl 1-benzoyl-2-cyanomethylpyrrolidine-2-carboxylate

Under nitrogen, a solution of lithium diisopropylamide (LDA) in tetrahydrofuran (THF) was generated in a 2 L three neck, round bottom flask, fitted with pressure-equaling addition funnel, as follows: Under a nitrogen atmosphere and while chilled in an ice bath, n-butyllithium (462 mL of 2.5 M in hexanes, 1.15 mol) was added dropwise to a magnetically stirred solution of diisopropylamine (124 g, 1.22 mol) in anhydrous THF (500 mL) over a 65 min period. The light-yellow LDA solution was stirred at 0° C. for 1 h.

In a 5 L 3-necked round bottom flask fitted with overhead stirrer and a nitrogen inlet, methyl 1-benzoylpyrrolidine-2-carboxylate (220 g, 0.943 mol) was slurried in anhydrous THF (500 mL) and was cooled to −77° C. (dry ice-acetone bath) under nitrogen. The LDA solution was cannulated into the methyl 1-benzoylpyrrolidine-2-carboxylate solution (maintained at −77° C.) over a period of 1 h. The resulting solution was stirred for 2 h at −77° C., during which time its color changed from yellow to orange. To this solution (maintained at −77° C.) was added a solution of bromoacetonitrile (146 g, 1.22 mol) in anhydrous THF (420 mL) via cannula over a 2 h period. The resulting orange-brown solution was stirred at −77° C. for 1 h and then allowed to warm to ambient temperature (overnight). The reaction was quenched by the addition of saturated aqueous ammonium chloride (600 mL). To facilitate phase separation, the brown, biphasic mixture was suction filtered, and the salts washed with t-butyl methyl ether (TBME) (˜300 mL). The organic layer was separated, and the aqueous phase was extracted with TBME (300 mL), suction filtered again to remove more solids, and extracted with TBME (600 mL) a second time. The combined organic phases were washed with 1 M hydrochloric acid (1.3 L) and half-saturated aqueous sodium chloride (1.3 L). The aqueous washes were extracted with TBME (100 mL) and the combined organic phases were dried over anhydrous sodium sulfate (165 g) and concentrated by rotary evaporation to give a black oil (249 g). This residue was partially dissolved in TBME (1.08 L), hexanes (270 mL) was added, and the mixture was stirred (overhead stirrer) for 1 h at room temperature and then allowed to stand, without agitation, overnight The TBME-hexanes solution was decanted away from the black tarry residue and passed through a column of silica gel (220 g) (5.5 cm i.d.×25 cm). The column was washed with an additional volume of TBME-hexanes (80:20, v/v) (2×600 mL), and the total eluate (˜1.9 L) was concentrated by rotary evaporation to give 196 g (76.4%) of methyl 1-benzoyl-2-cyanomethylpyrrolidine-2-carboxylate as a very viscous, amber oil (97.5% pure by HPLC).

### 1-Benzoyl-1,7-diazaspiro[4.4]nonan-6-one

Two identical solutions of methyl 1-benzoyl-2-cyanomethylpyrrolidine-2-carboxylate (32.3 g, 0.118 mol) in anhydrous methanol (˜75 mL) were cooled in ice water as concentrated sulfuric acid (19 mL, 0.34 mol) was cautiously added to each with stirring. These solutions were transferred under a nitrogen atmosphere to two Parr hydrogenation bottles (500 mL capacity), each containing 10 wt % palladium on carbon catalyst (13.6 g), using anhydrous methanol (125 mL) to facilitate each transfer. The Parr bottles were flushed with nitrogen and then each was attached to a Parr hydrogenation apparatus. The mixtures were each shaken under 50 psi hydrogen pressure at room temperature for 23 h (overnight). Each hydrogenation mixture was filtered through a pad of diatomaceous earth (25 g), and the filter cakes were each washed with methanol (250 mL). The combined pale-yellow filtrates (both reactions) were concentrated by rotary evaporation, producing an amber oil. This was cooled n an ice-water bath and carefully basified with saturated aqueous sodium bicarbonate (660 mL), followed by addition of solid potassium carbonate (125 g, 0.907 mol) in portions, giving a final pH of 9-10 (pH paper). This mixture was gently refluxed overnight (solids present) and cooled to ambient temperature. Dichloromethane (625 mL) and water (600 mL) were added and the mixture was stirred to dissolve all solids. The aqueous phase was separated and extracted with dichloromethane (2×150 mL, 3×100 mL). The combined dichloromethane phases were dried over sodium sulfate, filtered, and concentrated by rotary evaporation to give a beige solid. This solid was slurried in hot (near reflux) isopropyl acetate (105 mL), cooled to room temperature, and further cooled to 5° C. overnight The solids were filtered under a nitrogen purge, washed with isopropyl acetate (2×25 mL) and dried under vacuum at 50° C. for 9 h to give 37.8 g (65.3% yield) of 1-benzoyl-1,7-diazaspiro[4.4]nonan-6-one as an off-white solid (98.8% pure by HPLC).

### 1-Benzyl-1,7-diazaspiro[4.4]nonane

1-Benzoyl-1,7-diazaspiro[4.4]nonan-6-one (44.0 g, 0.18 mol) and anhydrous tetrahydrofuran (THF) (700 mL) were placed in a 3 L three neck, round bottom flask fitted with overhead stirrer, reflux condenser (with nitrogen inlet) and 1 L addition funnel (pressure-equalizing). This mixture was stirred under a nitrogen atmosphere as it was cooled to <10° C. in an ice-water bath. Lithium aluminum hydride (540 mL of 1 M solution in THF, 0.54 mol) was added dropwise to the continuously cooled mixture over a 51 min period, ultimately producing a very pale-yellow solution. The ice-water bath was then removed and the solution was stirred and heated (via heating mantle) under nitrogen at mild reflux for 21 h. The turbid mixture was diluted with THF (450 mL) and again cooled in an ice-water bath. The excess lithium aluminum hydride was decomposed by careful drop-wise addition of, in order, water (17 mL), 15% NaOH solution (17 mL), water (50 mL), and anhydrous sodium sulfate (50 g). This mixture was stirred and then filtered through a pad of diatomaceous earth (82 g), washing the filter cake with THF (3×100 mL). The filtrate was dried over anhydrous sodium sulfate, filtered, and concentrated by rotary evaporation. The solid was vacuum dried (73° C. for ˜0.5 h) to give 37.3 g (95.6%) of 1-benzyl-1,7-diazaspiro[4,4]nonane as a yellow oil (97% pure by HPLC).

### 1-Benzyl-7-(3-pyridinyl)-1,7-diazaspiro[4.4]nonane

A 1 L three neck, round bottom flask, fitted with heating mantle, addition funnel, vacuum take-off, and a condenser fitted with nitrogen inlet, was charged, in order, with the following reagents: sodium tert-butoxide (44.3 g, 0.461 mol), tris(dibenzylideneacetone) dipalladium(0) (6.04 g, 6.59 mmol), racemic-2,2′-bis(diphenylphosphino)-1,1′-binaphthyl (rac-BINAP) (8.75 g, 13.2 mmol) and a solution of 1-benzyl-1,7-diazaspiro[4.4]nonane (73.5 g of 97% purity, 0.330 mol) in toluene (285 mL). While the mixture was rapidly (magnetically) stirred, the flask was repeatedly evacuated and filled with nitrogen (4 cycles). The vacuum take-off was then replaced with a thermocouple thermometer and the mixture was stirred and heated at 65-75° C. while a solution of 3-bromopyridine (52.1 g, 0.330 mol) in toluene (150 mL) was added from the addition funnel over a 1 h period. The addition funnel was rinsed with toluene (25 mL). Because the increased viscosity of the mixture made magnetic stirring inefficient, the magnetic stir bar was replaced with an overhead stirrer. The mixture was stirred and heated at 62-72° C. for 4 h and cooled to ambient temperature overnight. The reaction mixture was then cooled in an ice-water bath and poured into a mixture of 10% aqueous sodium chloride (200 mL) and tert-butyl methyl ether (TBME) (300 mL). This biphasic mixture was filtered through a pad of diatomaceous earth (18 g), washing the filter cake with TBME (3×50 mL). The organic phase was separated, cooled in an ice-water bath and treated with 6 M hydrochloric acid (140 mL), causing precipitation of a tan, gummy solid. This biphasic mixture was filtered through a pad of diatomaceous earth (18 g), and the filter cake was washed with 3 M hydrochloric acid (50 mL). The aqueous phase was separated and cooled in an ice-water bath as TBME (500 mL), and then 50% aqueous sodium hydroxide (100 mL), were added drop-wise (with stirring) via addition funnel (final pH=13). The dark-brown TBME phase was removed and the alkaline aqueous layer was extracted with TBME (2×100 mL). The combined TBME phases were dried over anhydrous sodium sulfate, filtered, and passed through a column of silica gel (100 g), collecting the orange-yellow eluent. An additional volume of TBME (500 mL or more, as needed) was added to completely elute the product. The TBME was removed by rotary evaporation, and the residue was vacuum dried at 30° C. for 6 h, to give 81.9 g (84.7%) of 1-benzyl-7-(3-pyridinyl)-1,7-diazaspiro[4.4]nonane as a light-beige powder (mp 100-101° C., 97.8% pure by HPLC).

### 7-(3-pyridinyl)-1,7-diazaspiro[4.4]nonane

A 2 L three neck, round bottom flask fitted with heating mantle, magnetic stir bar, reflux condenser with nitrogen inlet, and two addition funnels (500 mL capacity), was charged with 10 wt % palladium on carbon catalyst (Degussa type, water content ˜50 wt %) (44.5 g) and absolute ethanol (365 mL) under a nitrogen atmosphere. This mixture was gently heated (near reflux) while a solution of 98% formic acid (128 g, 2.79 mol) in absolute ethanol (370 mL) was added dropwise viaone addition funnel and a solution of 1-benzyl-7-(3-pyridinyl)-1,7-diazaspiro[4.4]nonane (81.8 g) in absolute ethanol (420 mL) was simultaneously added dropwise via the other addition funnel. Heating was interrupted whenever gas evolution became vigorous. The addition was complete in a period of 110 min. Under a cone of nitrogen, the hot mixture (near reflux) was filtered through a pad of diatomaceous earth (50 g), washing the filter cake with hot methanol (6×100 mL). The filtrate was cooled and concentrated by rotary evaporation. The residue was vacuum dried at 60° C. (water bath) to give a viscous, amber oil, to which was added chloroform (220 mL) and 10% aqueous sodium chloride (220 mL). This mixture was cooled in an ice-water bath and made basic (pH ˜12) by the addition of 5 M aqueous sodium hydroxide (50 mL). After thorough mixing, the chloroform phase was separated and the aqueous phase was extracted with chloroform (50 mL). The combined light-yellow chloroform extracts were washed with 10% aqueous sodium chloride (2×100 mL). The aqueous washes were extracted with chloroform (50 mL), and the combined chloroform phases were dried over anhydrous sodium sulfate. Concentration by rotary evaporation and vacuum drying of the resulting residue (71° C. for ˜30 min) gave 53.9 g (95.2% yield) of 7-(3-pyridinyl)-1,7-diazaspiro[4,4]nonane as a viscous, amber oil (99.3% pure by HPLC). ¹H NMR (DMSO-d₆): δ 7.86 (d, 1H, J=2.8 Hz), 7.79 (d, 1H, J=4.6 Hz), 7.12 (dd, 1H, J=8.2 Hz), 6.81 (m, 1H), 3.31 (m, 2H), 3.18 and 3.12 (AB q, 2H, J=9.4 Hz), 2.85 (m, 2H), 2.29 (broad s, N—H), 1.90 (m, 2H), 1.73 (m, 2H), 1.69 (m, 2H); ¹³C NMR (DMSO-d₆): δ 143.52, 136.03, 133.56, 123.46, 117.08, 67.70, 58.26, 46.45, 45.48, 36.76, 35.55, 25.19.

### Example 3

### Preparation of 7-(3-Pyridinyl)-1,7-diazaspiro[4.4]nonane mono-succinate salt

In a 1 L round bottom flask, 7-(3-pyridinyl)-1,7-diazaspiro[4.4]nonane (53.8 g, 0.265 mol) was dissolved in methanol (150 mL). To this solution was added a hot solution of succinic acid (31.3 g, 0.265 mol) in methanol (250 mL), rinsing with methanol (50 mL) in the transfer. The resulting red-brown solution was concentrated on a rotary evaporator to give a viscous, amber syrup. This was dissolved in hot (near reflux) ethanol (124 mL), and the solution was treated drop-wise with acetone (750 mL) over a 70 min period to precipitate the salt. The mixture was then cooled in a refrigerator (5° C.) overnight. The solid was collected by suction filtration under a nitrogen purge, washed with acetone (3×50 mL), and dried in a vacuum oven (40° C. for 8 h, followed by 50° C. for 4 h) to give 7-(3-pyridinyl)-1,7-diazaspiro[4.4]nonane mono-succinate (73.7 g, 86.6%) as an off-white powder, mp 131.5-133° C. (99.2% (a/a) by HPLC). ¹H NMR (D₂O): δ 7.83 (d, 1H, J=5.3 Hz), 7.81 (d, 1H, J=2.3 Hz), 7.51 (dd, 1H, J=8.7 Hz), 7.37 (m, 1H), 3.47 (m, 2H), 3.65 and 3.41 (AB q, 2H, J=11.3 Hz), 3.32 (m, 2H), 2.25 (s, 2H, —CH₂— of succinic acid, indicating a mono-salt stoichiometry), 2.33 (m, 2H), 2.07 (m, 2H), 2.04 (m, 2H); ¹³C NMR (D₂O): δ 181.67 (C═O of succinic acid), 144.91, 130.04, 126.56, 125.94, 125.86, 72.16, 54.78, 46.08, 45.01, 33.58 (—CH₂— of succinic acid), 33.47, 32.66, 22.82; ES-MS: [M+H]⁺ at m/e 204, consistent with the molecular weight (203.3) of the free base.

### Example 4

### Preparation of 7-(3-pyridinyl)-1,7-diazaspiro[4.4]nonane di-oxalate mono-hydrate salt

Oxalic acid (0.256 g, 2.84 mmol) was dissolved in a mixture of tetrahydrofurn (THF) (3 mL) and ethanol (1.4 mL), assisted by stirring and heating. To this hot, stirring solution, near reflux, a hot solution of 7-(3-pyridinyl)-1,7-diazaspiro[4.4]nonane (0289 g, 1.42 mmol) in ethanol (2 mL) was added dropwise, with additional ethanol (2×2 mL, 0.5 mL) used in the transfer. To facilitate stirring of the resulting gummy mass, additional ethanol (4 mL) was added and the mixture was heated to reflux. Methanol (4 mL) was added and the mixture was heated to reflux and stirred to produce a granular solid. The mixture was stirred at room temperature and then concentrated on a rotary evaporator, affording an off-white solid with some yellow clumps. The solid was slurried in hot methanol (6 mL), stirred, and heated to reflux to give fine, off-white crystals in a light-yellow liquor. The mixture was cooled to room temperature and acetone (18 mL) was added dropwise over 25 min. The resulting mixture was cooled at 5° C. for 16 h. The solids were filtered under a cone of nitrogen on a small funnel and washed with cold acetone (6.5 mL). The material was dried in a vacuum oven at 50° C. for 5 h to give 0.421 g (73.7%) of a light-beige powder. A portion (0.362 g) of the batch was slurried in methanol (5 mL), stirred and heated to reflux, cooled to room temperature and chilled (refrigerated) at 5° C. for 16 h. The solids were filtered under a cone of nitrogen and washed with cold methanol (2×2 mL). The solids were dried in a vacuum oven at 50° C. for 4 h to give 0.338 g (93.4% recovery) of 7-(3-pyridinyl)-1,7-diazaspiro[4.4]nonane di-oxalate mono-hydrate as a light-beige powder (98.48% (a/a) by GC-FID; 99.66% (a/a) by LC-DAD) mp 200-201.5° C. Elemental analysis results were consistent with a di-oxalate mono-hydrate stoichiometry. ¹H NMR (D₂O): δ 7.87 (d, 1H), 7.81 (m, 1H), 7.65 (dd, 1H), 7.52 (m, 1H), 3.69 and 3.46 (AB q, 2H), 3.50 and 3.33 (m, 4H), 2.35 (m, 2H), 2.06 (m, 4H).

### Example 5

### Preparation of 7-(3-pyridinyl)-1,7-diazaspiro[4.4]nonane di-hydrochloride salt

7-(3-Pyridinyl)-1,7-diazaspiro[4.4]nonane (800 mg, 3.94 mmol) was dissolved in isopropanol (˜5 mL) and 2 mL of HCl in dioxane (4 M, ˜8 mmol) was added, followed by ethanol (0.5 mL). The mixture was cooled in a dry ice bath to give a sticky yellowish-white solid. Additional isopropanol (˜20 mL) was added and the mixture was heated at reflux. The white solid residue remained, and was filtered to give 468 g (43.2% yield) of 7-(3-pyridinyl)-1,7-diazaspiro[4.4]nonane di-hydrochloride as a white solid (mp 242-244° C.). ¹H NMR (CD₃OD): δ 8.20 (s, 1H), 8.10 (d, 1H), 7.85 (m, 1H), 7.75 (dd, 1H), 3.95 and 3.60 (AB q, 2H), 3.65 (m. 2H), 3.45 (m, 2H), 2.50 (m, 2H), 2.22 (m, 4H).

### Example 6

### Resolution of 7-(3-pyridinyl)-1,7-diazaspiro[4.4]nonane into its isomers

### Preparation of 7-(3-pyridinyl)-1,7-diazaspiro[4.4]nonane mono-(+)-di-O,O′-p-toluoyl-D-tartrate in 2-propanol

(+)-Di-O,O′-p-toluoyl-D-tartaric acid (D-DTTA) (0.97 g, 2.5 mmol) was added to a hot (near reflux) solution of 7-(3-pyridinyl)-1,7-diazaspiro[4.4]nonane (0.51 g, 2.5 mmol) in 2-propanol (15 mL). The mixture was heated to reflux as water (1.8 mL) was added drop-wise to give a light amber solution. The solution was cooled to ambient temperature, at which temperature it remained overnight. The solution was seeded, solids began to form, and the mixture was stirred at ambient temperature for 2.5 h. The white solids were filtered, washed with 2-propanol (10 mL) and dried under vacuum with air purge to give 1.27 g (86.0%) of a white powder (from which the free base was shown to have 53.1% ee by chiral HPLC on a Chiralpak AD® column, using 75:25 hexane/ethanol). The solid was slurried in refluxing ethanol (28 mL) and water (1 mL) was added dropwise. The solution was cooled to ambient temperature, seeded and allowed to sit overnight. The mixture was stirred at ambient temperature for 3.5 h, filtered, washed with ethanol (5 mL) and vacuum dried at 50° C. for 3 h to give 0.54 g (42.3% recovery) of a white powder (92.9% ee by chiral HPLC). The solid was slurried in refluxing ethanol (12 mL) and water (1.3 mL) was added dropwise. The solution was cooled, seeded and allowed to sit overnight at ambient temperature. The resulting solids were filtered, washed with ethanol (3 mL), and dried at 50° C. overnight to give 0.39 g (73.1% recovery) of a solid (99.0% ee by chiral HPLC). The solid was recrystallized from ethanol/water (8.6 mL: 1.0 mL), seeded, allowed to stand overnight, stirred for 3 h, filtered, washed with ethanol (2 mL) and dried at 50° C. for 3 h to give 0.30 g (75.3% recovery) of a white powder (99.9% ee by chiral HPLC, mp 182-183° C.). ¹H NMR (DMSO-d₆): δ 7.87 (m, 2H), 7.84 (d, 4H, —C₆H₄—, indicating a mono-salt stoichiometry), 7.32 (d, 4H, —C₆H₄— of acid moiety, indicating a mono-salt stoichiometry), 7.16 (dd, 1H), 6.82 (m, 1H), 5.63 (s, 2H, —CH(CO₂H)—O— of acid moiety, indicating a mono-salt stoichiometry), 3.60 (d, 1H), 3.38 and 3.25 (m, 5H), 2.38 (s, 6H, —CH₃ of acid moiety, indicating a mono-salt stoichiometry), 2.35 and 2.10 (m, 2H), 1.92 (m, 4H).

### Preparation of 7-(3-pyridinyl)-1,7-diazaspiro[4.4]nonane mono-(+)-di-O,O′-p-toluoyl-D-tartrate in ethanol

(+)-Di-O,O′-p-toluoyl-D-tartaric acid (1.94 g, 5.01 mmol) in hot ethanol (3 mL+additional 4 mL to wash) was added to a hot solution of 7-(3-pyridinyl)-1,7-diazaspiro[4.4]nonane (1.02 g, 5.01 mmol) in ethanol (1 mL). The solution was cooled to ambient temperature, seeded and allowed to stand overnight to give a syrup, that was concentrated in vacuo to a light yellow foam. 2-Propanol (29 mL) was added and the mixture was heated, seeded, and stirred for 3 h. The resulting solids were filtered, washed with 2-propanol (6 mL), and dried at 50° C. under vacuum with air bleed to give 2.63 g (89.0%) of an off-white-light yellow powder (532% ee by chiral HPLC). The salt was slurried in refluxing ethanol (65 mL) and water (1.7 mL) was added drop-wise. The solution was seeded and allowed to stand at ambient temperature. The resulting white solids were stirred at ambient temperature for 8 h, filtered, washed with ethanol (10 mL) and dried at 50° C. under vacuum with air bleed overnight to give 1.10 g (41.8% recovery) of a white powder (91.7% ee by chiral HPLC). The solid was dissolved in refluxing ethanol and water (1.8 mL) was added dropwise. The solution was cooled, seeded, and stirred for 3.5 h. The solids were filtered, washed with ethanol (5 mL) and dried to give 0.85 g (77.0% recovery) of solid (98.7% ee by chiral HPLC). The solid was slurried in refluxing ethanol (12.5 mL) and water (1.4 mL) was added drop-wise. The solution was cooled, seeded, and allowed to stand overnight. The resulting solids were filtered, washed with ethanol (3 mL) and dried at 50° C. with air bleed to give 0.67 g (78.5% recovery) of a white powder (99.9% ee by chiral HPLC; mp=183-184° C.).

### Preparation of 7-(3-pyridinyl)-1,7-diazaspiro[4.4]nonane mono-(+)-di-O,O′-p-toluoyl-D-tartrate in ethanol with 0.75 equivalent D-DTTA

(+)-Di-O,O′-p-toluoyl-D-tartaric acid (3.69 g, 9.55 mmol) was added to a hot (50° C.) solution of 7-(3-pyridinyl)-1,7-diazaspiro[4.4]nonane (2.59 g, 12.7 mmol) in ethanol (25 mL). The solution was heated to near boiling and held at that temperature for 5 min. Small particles began to come out of solution and the mixture was cooled to ambient temperature and stirred for 2 h. The resulting solids were collected by filtration, washed with ethanol (10 mL), and dried for 10 min under nitrogen to give 2.87 (76.4%) of white crystals (94% ee by chiral HPLC).

### Liberation of free base from 7-(3-pyridinyl)-1,7-diazaspiro[4.4]nonane mono-(+)-di-O,O′-p-toluoyl-D-tartrate

To a sample (0.50 g, 0.84 mmol) of the 7-(3-pyridinyl)-1,7-diazaspiro[4.4]nonane mono-(+)-di-O,O′-p-toluoyl-D-tartrate was added 5M sodium hydroxide (3 mL) and water (5 mL). The mixture was stirred at ambient temperature overnight, and chloroform was added to form a suspension. The alkaline layer was separated and extracted with chloroform (3×10 mL). The combined chloroform extracts were washed with water (15 mL), dried over anhydrous sodium sulfate, filtered and concentrated to give 0.17 g of an amber oil (quantitative yield). The retention time of this sample on chiral HPLC corresponded to the longer (9.1 min) of the two peaks characteristic of the racemate (Chiralpak AD® column, using 75:25 hexane/ethanol). Free base 7-(3-pyridinyl)-1,7-diazaspiro[4.4]nonane liberated from a sample of the mono-(+)-di-O,O′-p-toluoyl-D-tartrate salt was analyzed by chiral HPLC, which showed 0.13% of the 1^{st} eluting compound (RT 8.3 min) and 99.87% of the 2^{nd} eluting compound (RT 9.2 min).

### Preparation of 7-(3-pyridinyl)-1,7-diazaspiro[4.4]nonane mono-(−)-di-O,O′-p-toluoyl-L-tartrate

(−)-Di-O,O′-p-toluoyl-L-tartaric acid (L-DTTA) (1.90 g, 4.92 mmol) in hot ethanol (3 mL+additional 4 mL to wash) was added to a hot solution of 7-(3-pyridinyl)-1,7-diazaspiro[4.4]nonane (1.00 g, 4.92 mmol) in ethanol (3 mL). The solution was cooled to ambient temperature, allowed to stand overnight and concentrated to give a light yellow/off-white foam. The foam was heated to reflux in 2-propanol (29 mL) to give an oily deposit, which became a solid upon cooling and stirring at ambient temperature for 2 h. The solids were filtered, washed with 2-propanol (6 mL) and dried under vacuum at 50° C. with an air bleed to give 2.60 g (89.5%) of a white/off-white solid (from which the free base was shown to have 49.5% ee by chiral HPLC on a Chiralpak AD® column, using 75:25 hexane/ethanol).

The solid was slurried in refluxing ethanol (65 mL), and water (1.5 mL) was added dropwise. The solution was cooled to ambient temperature and allowed to stand for 2 days. The resulting white solids were filtered, washed with ethanol (10 mL) and dried at 50° C. overnight with an air purge to give 1.03 g (39.8% recovery) of a white powder (90.7% ee by chiral HPLC). The solid was dissolved in refluxing ethanol (23 mL), and water (1.9 mL) was added drop-wise. The solution was cooled to ambient temperature, allowed to sit overnight, and stirred at ambient temperature for 3.5 h. The solids were filtered, washed with ethanol (5 mL) and dried at 50° C. for 3 h to give 0.77 g (74.6% recovery) of a white powder (99.3% ee by chiral HPLC, mp 182-183° C.). ¹H NMR (DMSO-d₆): δ 7.88 (m, 2H), 7.84 (d, 4H, —C₆H₄— of acid moiety, indicating a mono-salt stoichiometry), 7.32 (d, 4H, —C₆H₄— of acid moiety, indicating a mono-salt stoichiometry), 7.16 (dd, 1H), 6.84 (m, 1H), 5.64 (s, 2H, —CH(CO₂H)—O— of acid moiety, indicating a mono-salt stoichiometry), 3.62 (d, 1H), 3.38 and 3.28 (m, 5H), 2.38 (s, 6H, —CH₃ of acid moiety, indicating a mono-salt stoichiometry), 2.35 and 2.10 (m, 2H), 1.90 (m, 4H).

### Liberation of free base from 7-(3-pyridinyl)-1,7-diazaspiro[4.4]nonane mono-(−)-di-O,O′-p-toluoyl-L-tartrate

To a sample (0.43 g, 0.74 mmol) of 7-(3-pyridinyl)-1,7-diazaspiro[4.4]nonane mono-(−)-di-O,O′-p-toluoyl-L-tartrate was added 5M sodium hydroxide (3 mL) and water (5 mL). The mixture was stirred at ambient temperature overnight, and chloroform was added to form a suspension. The alkaline layer was separated and extracted with chloroform (3×10 mL). The combined chloroform extracts were washed with water (15 mL), dried over anhydrous sodium sulfate, filtered and concentrated to give 0.15 g of an amber oil (quantitative yield). The retention time of this sample on chiral HPLC corresponded to the shorter (7.96 min) of the two peaks characteristic of the racemate (Chiralpak AD® column, using 75:25 hexane/ethanol). Free base 7-(3-pyridinyl)-1,7-diazaspiro[4.4]nonane liberated from a sample of the mono-(−)-di-O,O′-p-toluoyl-L-tartrate salt was analyzed by chiral HPLC, which showed 100% of the 1^{st} eluting compound (RT 8.1 min) and none (below detection limit) of the 2^{nd} eluting compound (which eluted at ˜9.1 min in samples of lesser purity).

### Second generation procedure for preparation of the diastereomeric 7-(3-pyridinyl)-1,7-diazaspiro[4.4]nonane mono-di-O,O′-p-toluoyltartrate salts

(+)-Di-O,O′-p-toluoyl-D-tartaric acid D-DTTA (9.9 g, 26 mmol) was added to a hot (near reflux) solution of racemic 7-(3-pyridinyl)-1,7-diazaspiro[4.4]nonane (7.5 g of 89%, 33 mmol) in ethanol (75 mL) and the solution was stirred for 30 min. The resulting suspension was cooled to 20-25° C. and stirred for 2 h. The solids were filtered, washed with ethanol (2×5 mL), and dried at 60° C. under vacuum to give 8.1 g of white powder (93% ee by chiral HPLC). The powder was recrystallized from ethanol (110 mL) and water (12 mL) by refluxing the mixture for 15 minutes to give a nearly clear solution, cooling over 2 h to ambient temperature, and stirring for 2 h. The solids were filtered, washed with ethanol (2×5 mL), and dried in a vacuum oven for 2-3 h to give 6.7 g (69% of theoretical) of a powder (99.3% ee by chiral HPLC).

The combined filtrates from the above resolution were concentrated and the resulting residue was made basic with 6 N sodium hydroxide. Extraction with chloroform gave, after concentration, 3.5 g of the free base (89% ee by chiral HPLC). This was dissolved in ethanol, (−)-Di-O,O′-p-toluoyl-D-tartaric acid (L-DTTA) (4.9 g, 0.013 mmol) was added, and the mixture was healed at gentle reflux for 30 min. The resulting thick suspension was cooled and stirred at ambient temperature for 3 h. The solids were filtered, washed with ethanol (2×5 mL), and dried in a vacuum oven for 2-3 h to give 4.6 g (47% of theoretical) of a white powder (99.6% ee by chiral HPLC).

### Example 7

### Determination of Absolute Configuration for the R-Isomer by Single Crystal X-ray

### Preparation of R-7-(3-pyridinyl)-1,7-diazospiro[4.4]nonane mono p-hydroxybenzoate

A solution of the earlier eluting enantiomer of 7-(3-pyridinyl)-1,7-diazaspiro[4.4]nonane (liberated, as in example 6, from the mono-(−)-di-O,O′-p-toluoyl-L-tartaric acid salt) (0.20 g, 0.98 mmol) in acetone (3 mL) was treated with p-hydroxybenzoic acid (0.15 g, 1.1 mmol). A thick precipitate formed, and the mixture was heated at 60° C. for 5 min and cooled to ambient temperature. Methanol (1 mL) was added and the mixture stood for 6 h at ambient temperature. The solid was filtered and dried to give 0.26 g (76% yield) of white powder (mp 136-138° C.).

### Preparation of R-7-(3-pyridinyl)-1,7-diazospiro[4.4]nonane mono p-chlorobenzoate

To a room-temperature, stirred solution of 7-(3-pyridinyl)-1,7-diazospiro[4.4]nonane (1.222 g, 6.01 mmol; earlier eluting enantiomer, isolated from the (−)-di-O,O′-p-toluoyl-L-tartrate salt) in acetone (25 mL) was added p-chlorobenzoic acid 0.941 g (6.01 mmol) in small portions. When about half of the acid had been added, crystalline solids started to precipitate. On completion of addition of the acid, additional acetone was added (20 mL), and the mixture was heated to near boiling until almost complete solution was obtained. Heating was discontinued and the solution was allowed to cool to ambient temperature (21.5° C.) without stirring. Crystallization was allowed to proceed for 16 h. Collected crystals were dried in vacuum oven at 80° C. for 2 h, affording 1.695 g of salt (76.7%) with a melting point of 144-146° C. (Fisher-Johns Apparatus). ¹H-NMR (D₂O): δ 7.72 (s & d, 2H), 7.62 (d, 2H), 7.26 (d, 2H), 7.15 (dd, 1H), 6.88 (d, 1H), 3.52 (d, 1H), 3.30 (m, 5H), 2.23 (m, 2H), 2.03 (m, 4H).

### Determination of Absolute Configuration by X-Ray Diffraction

Two attempts were made to establish the absolute configuration or the enantiomers of 7-(3-pyridinyl)-1,7-diazaspiro[4.4]nonane. Both attempts used the earlier eluting enantiomer (chiral HPLC analysis), which corresponds to the material derived from the mono-(−)-di-O,O′-p-toluoyl-L-tartaric acid salt. In the first attempt, 7-(3-pyridinyl)-1,7-diazaspiro[4.4]nonane mono-p-hydroxybenzoate, of which a crystal of suitable size had been obtained by recrystallization from acetone, was subjected to x-ray crystallographic analysis. Single crystal data X-ray data was collected using a Bruker SMART CCD diffractometer equipped with an Oxford “Cryostream” LT temperature apparatus operating at T=170K. A suitable crystal (0.3×0.3×0.2 mm) was chosen and mounted on a glass fiber using grease. Data were measured using omega scans of 0.3° per frame for 30 seconds, such that a full-sphere was collected. The first 50 frames were recollected at the end of data collection to monitor for decay. Cell parameters were retrieved using SMART [1] software and refined using SAINT [2] on all observed reflections. Data reduction was performed using the SAINT software, which corrects for LP and decay.

The resulting data fitted the S absolute configuration (using the Cahn-Ingold-Prelog convention) of 7-(3-pyridinyl)-1,7-diazaspiro[4.4]nonane somewhat better than the R absolute configuration, although the crystallographer noted that the standard deviation in the data made the determination of absolute structure unreliable. One potential reason for this is the lack of a heavy atom internal reference in the p-hydroxybenzoate salt.

In the second attempt, 7-(3-pyridinyl)-1,7-diazaspiro[4.4]nonane mono-p-chlorobenzoate was used. The p-chlorobenzoate counterion has the advantage of containing a heavy atom (chlorine) which serves as an internal reference in the crystal lattice. Single crystal data X-ray data was collected using a Nonius Kappa CCD diffractometer equipped with a fine-focus sealed tube, Mo Kα, radiation source. Apparatus, parameters and results are summarized in Tables 4 and 5 below.

TABLE 4
Sample and crystal data for Compound A, 4-Chlorobenzoate

| Project/Programme/F.S. | P858 | |
|---|---|---|
| Chemist's labbook | OS-352-6-13 | |
| X-ray labbook | PHX-08-003 | |
| Crystallization labbook | OS-352-6-13 Å | |
| Crystallization solvents | Acetonitrile | |
| Crystallization method | Slow evaporation | |
| Empirical formula | C₁₉H₂₂N₃O₂Cl | |
| Formula weight | 359.85 | |
| Temperature | 180(1) K | |
| Wavelength | 0.71069 Å | |
| Crystal size | 0.46 × 0.10 × 0.01 mm | |
| Crystal habit | Colourless Lath | |
| Crystal system | Orthorhombic | |
| Space group | P2₁2₁2₁ | |
| Unit cell dimensions | a = 8.3995(3) Å | α = 90° |
| | b = 20.2295(6) Å | β = 90° |
| | c = 20.9884(8) Å | γ = 90° |

| | |
|---|---|
| Volume | 3566.3(2) Å³ |
| Z | 8 |
| Denisty (calculated) | 1.340 Mg/m³ |
| Absorption coefficient | 0.232 mm⁻¹ |
| F(000) | 1520 |

TABLE 5
Data collection and structure refinement
for Compound A, 4-Chlorobenzoate

| | |
|---|---|
| Diffractometer | Nonius Kappa CCD |
| Radiation source | Fine-focus sealed tube, Mo Kα |
| Data collection method | Narrow frame ω and φ scans |
| Theta range for data | 3.54 to 22.42° |
| collection | |
| Index ranges | −8 ≦ h ≦ 8, −21 ≦ k ≦ |
| | 21, −22 ≦ I ≦ 22 |
| Reflections collected | 13846 |
| Independent reflections | 4520 [R(int) = 0.0755] |
| Coverage of independent | 98.8% |
| reflections | |
| Variation in check reflections | N/A |
| Max. and min. transmission | 0.9977 and 0.9008 |
| Structure solution technique | direct |
| Structure solution program | SHELXS-97 (Sheldrick, 1990) |
| Refinement technique | Full-matrix least-squares on F² |
| Refinement program | SHELXL-97 (Sheldrick, 1997) |
| Function minimized | Σ w(Fₒ²-F_{c}²)² |
| Data/restraints/parameters | 4520/0/464 |
| Goodness-of-fit on F² | 1.098 |
| Δ/σₘₐₓ | 0.000 |
| Final R indices | |
| 3659 data; I > 2σ(I) | R1 = 0.0494, wR2 = 0.1176 |
| All data | R1 = 0.0700, wR2 = 0.1287 |
| Weighting scheme | calc w = 1/[σ²(Fₒ²) + |
| | (0.0644 P)² + 0.6410 P] |
| | Where P = (Fₒ² + 2F_{c}²)/3 |
| Absolute structure parameter | 0.00(9) |
| Largest diff. peak and hole | 0.211 and −0.204 e Å−3 |
| Refinement summary: | |
| Ordered Non-H atoms, XYZ | Freely refining |
| Ordered Non-H atoms, U | Anisotropic |
| H atoms (on carbon), XYZ | Idealized positions riding on |
| | attached atoms |
| H atoms (on carbon), U | Appropriate multiple of U(eq) for |
| | bonded atom |
| H atoms (on heteroatoms), XYZ | Idealized positions riding on |
| | attached atoms |
| H atoms (on heteroatoms), U | Appropriate multiple of U(eq) for |
| | bonded atom |
| Disordered atoms, OCC | N/A |
| Disordered atoms, XYZ | N/A |
| Disordered atoms, U | N/A |

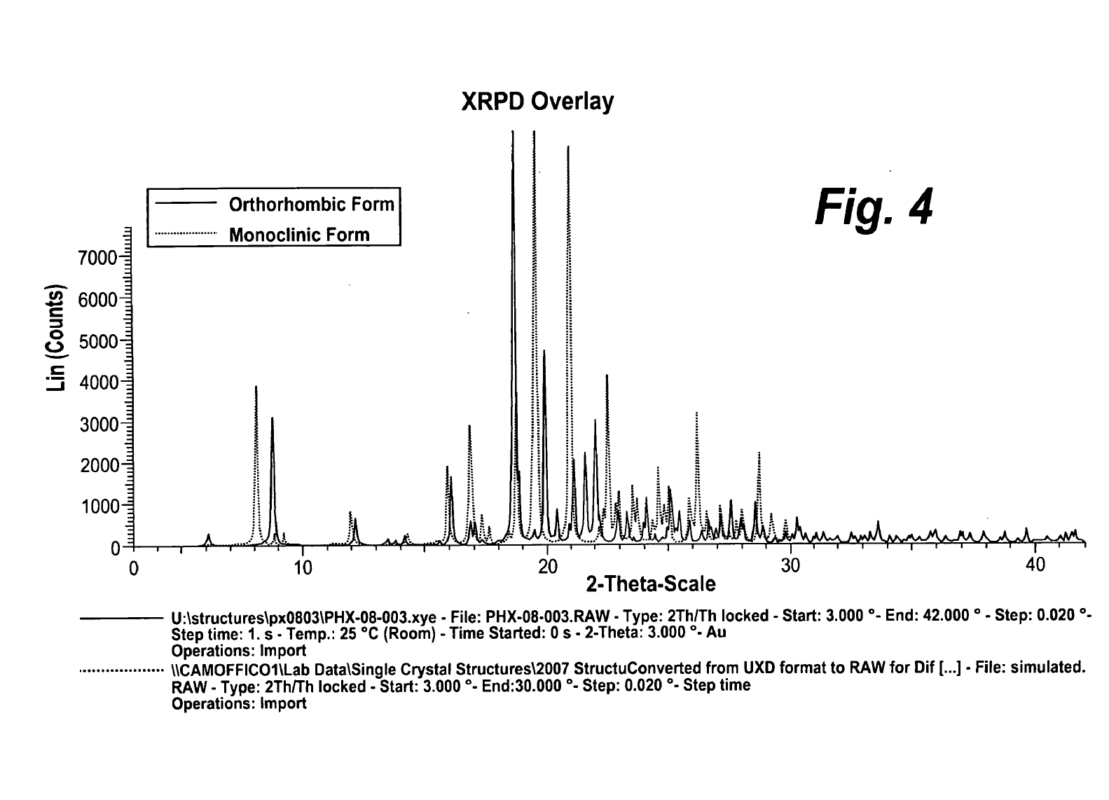

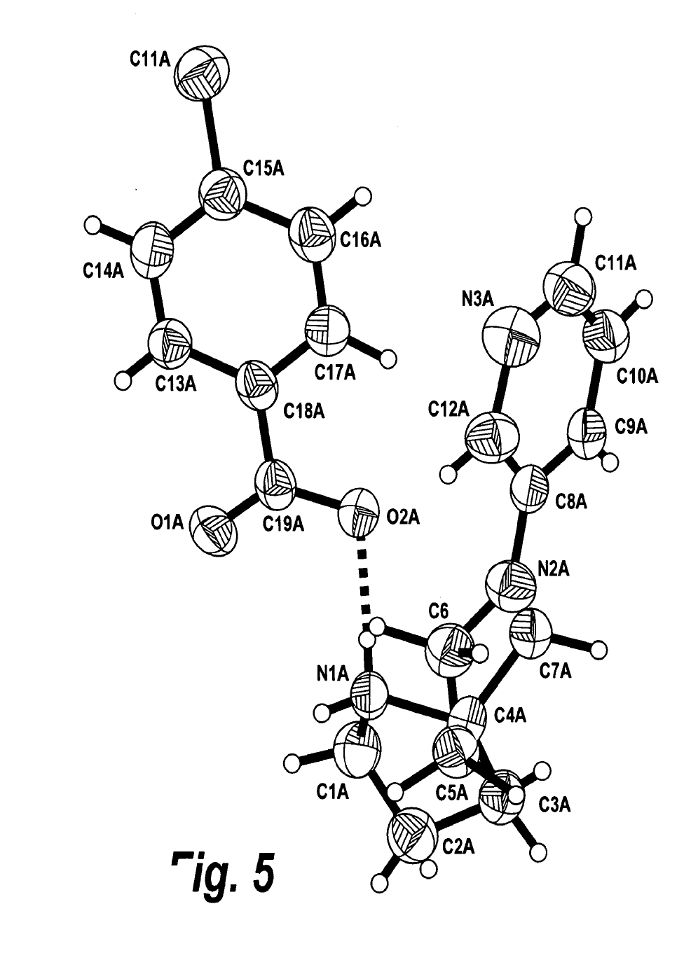

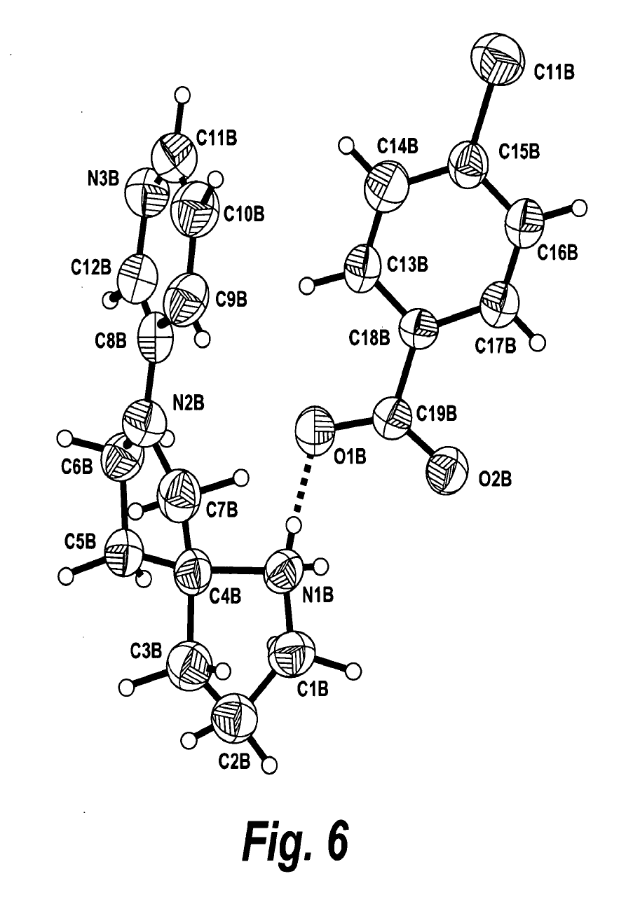

The single crystal X-ray structure of the sample was determined using crystalline material obtained by the recrystallization of sample E00301 (as supplied) from acetonitrile via slow evaporation. The structure determined was orthorhombic, space group P212121, with two independent molecules in the asymmetric unit. The structure previously determined (with sample E00301) was found to be monoclinic, space group P21, meaning that at least two polymorphs of 7-(3-pyridinyl)-1,7-diazaspiro[4.4]nonane mono-p-chlorobenzoate exist. A comparison of the calculated XRPDs for the two crystalline forms is shown in FIG. 4. The absolute stereochemistry was determined as R from consideration of the Flack parameter, which was determined to be 0.00 (9). Furthermore, the determination of the absolute stereochemistry using Bayesian statistics on the Bijvoet pair differences resulted in a probability of the stereochemistry at the chiral center being R as 1.00 and that of the chiral center being S as 0.00, which is in agreement with the assignment from the Flack parameter. Three dimensional images of the two molecules in the asymmetric unit are shown in FIGS. 5 and 6.

Thus, the 7-(3-pyridinyl)-1,7-diazaspiro[4.4]nonane enantiomer derived from the mono-(−)-di-O,O′-p-toluoyl-L-tartaric acid salt, also characterized by the shorter retention time on chiral HPLC, has the R absolute configuration. It follows, therefore, that the other enantiomer (i.e., that derived from the mono-(+)-di-O,O′-p-toluoyl-D-tartaric acid salt, also characterized by the longer retention time on chiral HPLC, has the S absolute configuration.

### Example 8

### Dioxalate salt of (S)-7-(3-pyridinyl)-1,7-diazospiro[4.4]nonane

A solution of (S)-7-(3-pyridinyl)-1,7-diazaspiro[4.4]nonane (0.15 g, 0.71 mmol) in methanol (0.5 mL) was treated with a solution of oxalic acid (0.13 g, 1.4 mmol) in hot methanol (1 mL+additional 2 mL to wash). The resulting solution was concentrated in vacuo to an oil, and acetone was added to give a gummy semi-solid that became a solid upon scratching. The mixture was stirred at ambient temperature overnight. The solids were filtered, washed with acetone (2×5 mL), and dried at 45° C. for 4 h in a vacuum oven to give 0.23 g (83% yield based on dioxalate) of (S)-7-(3-pyridinyl)-1,7-diazospiro[4.4]nonane dioxalate as a white solid (mp 135-138.5° C.). ¹H NMR (D₂O): δ 7.84 (d, 1H), 7.81 (d, 1H), 77.60 (dd, 1H), 7.67 (m, 1H), 3.68 and 3.44 (AB q, 2H), 3.49 and 3.33 (m, 4H), 2.35 (m, 2H), 2.06 (m, 4H).

### Example 9

### p-Hydroxybenzoate salt of (S)-7-(3-pyridinyl)-1,7-diazospiro[4.4]nonane

A solution of (S)-7-(3-pyridinyl)-1,7-diazaspiro[4.4]nonane (0.15 g, 0.76 mmol) in acetone (1 mL) was treated with a warm solution of 4-hydroxybenzoic acid (0.10 g, 0.76 mmol) in acetone (1 mL+additional 3 mL to wash). The resulting gummy white residue was dissolved in methanol with heating. The mixture was concentrated in vacuo to give a white semisolid which was treated with 2-propanol (2-3 mL). The mixture was stirred at ambient temperature overnight The white solids were filtered under nitrogen, washed with 2-propanol (5 mL), and dried at 45° C. for 4 h in a vacuum oven to give 0.18 g (70% yield) of (S)-7-(3-pyridinyl)-1,7-diazospiro[4.4]nonane p-hydroxybenzoate as an off-white solid (mp 136-138° C.). ¹H NMR (D₂O): δ 7.89 (m, 2H), 7.77 (distorted d, 2H, —C₆H₄— of acid moiety, indicating a mono-salt stoichiometry), 7.31 (dd, 1H), 7.09 (m, 1H), 6.88 (distorted d, 2H, —C₆H₄— of acid moiety, indicating a mono-salt stoichiometry), 3.70 and 3.40 (AB q, 2H), 3.55 and 3.45 (m, 4H), 2.40 (m, 2H), 2.18 (m, 4H).

### Example 10

### (R)-Mandelate salt of (S)-7-(3-pyridinyl)-1,7-diazospiro[4.4]nonane

A solution of (S)-7-(3-pyridinyl)-1,7-diazaspiro[4.4]nonane (0.13 g, 0.63 mmol) in 2-propanol (0.5 mL) was treated with a warm solution of (R)-(−)-mandelic acid (0.10 g, 0.63 mmol) in 2-propanol (1 mL+additional 1 mL to wash). Isopropyl acetate (7 mL) was added dropwise to the colorless solution, producing no cloudiness. The mixture was concentrated in vacuo to give a light yellow gum that was dried overnight at 60° C. in a vacuum oven. Acetone (5 mL) was added to dissolve most of the resulting yellow gum and upon standing at ambient temperature, crystals began to form. The mixture was refrigerated for 3-4 h and the resulting crystalline solid was filtered under nitrogen and washed with acetone (4 mL). The solids were dried under vacuum at 60° C. for 2 h to give 0.18 g (79% yield) of (S)-7-(3-pyridinyl)-1,7-diazospiro[4.4]nonane (R)-mandelate as a white granular powder (mp 138-149° C.). ¹H NMR (D₂O): δ 7.93 (m, 2H), 7.10 (m, 5H, —C₆H₅ of acid moiety, indicating a mono-salt stoichiometry), 7.39 (m, 1H), 7.22 (m, 1H), 4.98 (s, 1H, —CH(OH)— of acid moiety, indicating a mono-salt stoichiometry), 3.75 (d, 1H), 3.57 and 3.47 (m, 5H), 2.42 (m, 2H), 2.20 (m, 4H).

### Example 11

### Hydrochloride salt of (S)-7-(3-pyridinyl)-1,7-diazospiro[4.4]nonane

A solution of (S)-7-(3-pyridinyl)-1,7-diazaspiro[4.4]nonane (0.14 g, 0.68 mmol) in absolute ethanol (1 mL) was treated with concentrated (12 M) HCl (118 μL, 1.37 mmol). The solution was concentrated in vacuo and dried under vacuum at 60° C. overnight. The resulting white solid was treated with acetone (3 mL), and the mixture was stirred at ambient temperature for 4 h and refrigerated overnight. The solid was filtered under nitrogen and washed with acetone (3 mL). The light yellow solids were dried under vacuum at 50° C. for 20 h to give 0.17 g (90% yield) of (S)-7-(3-pyridinyl)-1,7-diazospiro[4.4]nonane hydrochloride as a hygroscopic yellow powder. ¹H NMR (D₂O): δ 8.00 (s, 1H), 7.97 (m, 1H), 7.69 (dd, 1H), 7.55 (m, 1H), 3.82 and 3.57 (AB q, 2H), 3.65 (m. 2H), 3.47 (m, 2H), 2.50 (m, 2H), 2.22 (m, 4H).

### Example 12

### Benzoate salt of (S)-7-(3-pyridinyl)-1,7-diazospiro[4.4]nonane

A solution of (S)-7-(3-pyridinyl)-1,7-diazaspiro[4.4]nonane (1.48 g, 7.29 mmol) in isopropyl acetate (10 mL) was treated with benzoic acid (0.89 g, 7.3 mmol) to give a solution. Solids began to separate, additional isopropyl acetate (5 mL) was added, and the mixture was stirred at ambient temperature overnight. The salt was collected by filtration under nitrogen and dried for 5 h in a vacuum oven at 75° C. to give 2.23 g (93.9% yield) of (S)-7-(3-pyridinyl)-1,7-diazospiro[4.4]nonane benzoate as a white solid (mp 115-115.5° C.). ¹H NMR (D₂O): δ 7.75 and 7.67 (m, 4H), 7.30 (m, 3H, —C₆H₅ of acid moiety, indicating a mono-salt stoichiometry), 7.12 (m, 1H), 6.90 (m, 1H), 3.55 (d, 1H), 3.38 and 3.24 (m, 5H), 2.24 (m, 2H), 1.99 (m, 4H).

### Example 13

### Benzoate salt of (R)-7-(3-pyridinyl)-1,7-diazospiro[4.4]nonane

A solution of (R)-7-(3-pyridinyl)-1,7-diazaspiro[4.4]nonane (1.67 g, 8.20 mmol) in isopropyl acetate (10 mL) was treated with benzoic acid (1.00 g, 8.20 mmol) to give a solution. Solids began to separate, additional isopropyl acetate (5 mL) was added and the mixture was stirred at ambient temperature overnight. The salt was collected by filtration under nitrogen and dried for 5 h in a vacuum oven at 75° C. to give 2.44 g (91.3% yield) of (R)-7-(3-pyridinyl)-1,7-diazospiro[4.4]nonane benzoate as a white solid (mp 115-115.5° C.). ¹H NMR (D₂O): δ 7.75 and 7.67 (m, 4H), 7.37 (m, 1H, —C₆H₅ of acid moiety, indicating a mono-salt stoichiometry), 7.30 (m, 2H, —C₆H₅ of acid moiety, indicating a mono-salt stoichiometry), 7.15 (m, 1H), 6.91 (m, 1H), 3.54 (d, 1H), 3.40 and 3.28 (m, 5H), 2.23 (m, 2H), 2.00 (m, 4H).

### Example 14

### Hemigalactarate (hemimucate) salt of (R)-7-(3-pyridinyl)-1,7-diazospiro[4.4]nonane

A solution of (R)-7-(3-pyridinyl)-1,7-diazaspiro[4.4]nonane (0.21 g, 1.03 mmol) in methanol (3 mL) was treated with mucic (galactaric acid) (0.12 g, 0.51 mmol) to give a thick precipitate. The mixture was heated to reflux and water (0.3 mL) was added to give a clear solution which was then cooled to ambient temperature over 1 h. The cooled solution was left overnight at ambient temperature. The precipitated solids were filtered and dried to give 0.18 g (60% yield) of (R)-7-(3-pyridinyl)-1,7-diazospiro[4.4]nonane hemigalactarate as a white plates.

### Example 15

### p-Bromobenzoate salt of (R)-7-(3-pyridinyl)-1,7-diazospiro[4.4]nonane

To a warm (60° C.), stirred solution of (R)-7-(3-pyridinyl)-1,7-diazaspiro[4.4]nonane (1.23 g, 6.03 mmol) in isopropyl acetate (15 mL) was added p-bromobenzoic acid (1.21 g, 6.03 mmol) in one portion. In a few minutes, a thick precipitate formed, and the mixture was cooled to ambient temperature and stirred overnight. The solid was collected by suction filtration and vacuum dried (75° C. for 3 h) to give 2.27 g (93.3% yield) of a yellow granular solid (mp 138-144° C.). ¹H-NMR (CDCl₃):): δ 8.93 (broad singlet 2H, ⁺NH₂), 7.95 (s & d, 2H), 7.73 (d, 2H), 7.45 (d, 2H), 7.03 (dd, 1H), 6.71 (d, 1H), 3.72 (d, 1H), 3.57 (dd, 1H), 3.31 (m, 4H), 2.50 (m, 1H), 2.15 (m, 1H), 1.98 (m, 4H)

### Example 16

### Synthesis of (R)- and (S)-7-(3-pyridinyl)-1,7-diazaspiro[4.4]nonane via Separation of a Diastereomeric Intermediate

### N-Benzoyl-2-allylproline

N-Benzoyl-2-allylproline was generated by basic hydrolysis of the corresponding methyl ester (Sato et al., Heterocycles 37(1): 245 (1994)).

### N-Benzoyl-2-allylproline (R)-α-methylbenzyl amide

To a solution of N-benzoyl-2-allylproline (14.9 g, 57.0 mmol) in ether (100 mL) was added thionyl chloride (8.5 g, 72 mmol) and catalytic DMF (˜0.1 mL). The mixture was stirred overnight at ambient temperature, and then concentrated to dryness. The residue was dissolved in dichloromethane (100 mL), and the resulting solution added dropwise to an ice-cooled solution of (R)-α-methylbenzylamine (7.3 g, 60 mmol), triethylamine (14 mL, 100 mmol) and 4-(N,N-dimethylamino)pyridine (catalytic, 100 mg) in dichloromethane (250 mL). After stirring at ambient temperature overnight, the reaction was stirred vigorously while adding water (50 mL). After stirring for 15 min, the layers were separated, and the organic layer washed successively with 10% aqueous hydrochloric acid, water, 10% aqueous potassium carbonate, and brine (50 mL each). The organic layer was dried over anhydrous sodium sulfate, filtered and concentrated. The residue was purified by column chromatography on silica gel with a hexane-ethyl acetate gradient (0-50% ethyl acetate) to give two diastereomeric amides. The higher R_{f} diastereomer (2:1 hexane/ethyl acetate) was obtained as an oil (4.7 g, 45% of theoretical), while the more polar diastereomer was obtained as a crystalline solid (4.3 g, 41% of theoretical). NMR (CDCl₃): Less polar diastereomer: 1.55 (d, 3H); 1.7 (m, 2H); 1.85-2.0 (m, 2H); 2.75 (m, 1H); 2.95 (dd, 1H); 3.25-3.45 (m, 3H); 5.15 (q, 1H); 5.3 (m, 2H); 5.85 (m, 1H); 7.4 (m, 10H); 8.6 (br d, 1H). More polar diastereomer: 1.55 (d, 3H); 1.8 (m, 2H); 1.85-2.0 (m, 2H); 2.7 (m, 1H); 2.85 (dd, 1H); 3.3 (dd, 1H); 3.45 (m, 2H); 5.1 (q, 1H); 5.2 (m, 2H); 5.8 (m, 1H); 7.4 (m, 10H); 8.5 (br d, 1H).

### (S)-7-(3-pyridinyl)-1,7-diazaspiro[4.4]nonane dihydrochloride

A solution of the more polar diastereomer of N-benzoyl-2-allylproline (R)-α-methylbenzyl amide (4.00 g, 10.9 mmol) in dichloromethane (100 mL), cooled to −78° C., was treated with ozone enriched oxygen for 25 min. The resulting blue solution was purged with nitrogen to remove excess ozone and then treated with dimethyl sulfide (0.5 mL). The mixture was stirred for 4 h, gradually warming to ambient temperature. The mixture was then treated with triethylsilane (12 mL), followed by rapid drop-wise addition of trifluoroacetic acid (8 mL), and stirred overnight under nitrogen atmosphere. The reaction mixture was concentrated to dryness, and the residue was dissolved in dichloromethane (100 mL). This solution was washed successively with 10% aqueous potassium carbonate, water and brine (25 mL each). The organic layer was dried with anhydrous sodium sulfate, filtered and concentrated. The residue was purified by column chromatography on silica gel with a methanol/dichloromethane gradient (0-10% methanol). The product fractions thus obtained were still contaminated with excess triethylsilane, and were re-chromatographed with a hexane/ethyl acetate gradient elution (0-50% ethyl acetate) to give a solid product. This was recrystallized from hot hexane-ethyl acetate to give 1.6 g of crystalline 1-benzoyl-7-((R)-α-methylbenzyl)-1,7-diazaspiro[4.4]nonan-6-one (42%). NMR (CDCl3): 1.65 (d, 3H); 1.8-1.95 (m, 3H); 2.05-2.2 (m, 1H); 2.25-2.35 (m, 1H); 2.65-2.75 (m, 1H); 2.85-2.95 (m, 1H); 3.5-3.65 (m, 3H); 5.55 (q, 1H); 7.2-7.4 (m, 8H); 7.55 (m, 2H). MS: M+H=349

A solution of the above 1-benzoyl-7-((R)-α-methylbenzyl)-1,7-diazaspiro[4.4]nonan-6-one (1.6 g, 4.6 mmol) in dry tetrahydrofuran (THF) (50 mL) was added dropwise to a suspension of lithium aluminum hydride (0.480 g, 12.9 mmol) in dry THF (25 mL) with ice bath cooling. After 30 min of stirring with ice bath cooling, the bath was removed and the reaction mixture was heated under reflux overnight. It was then cooled in an ice bath and diluted with ether (50 mL). The cooled mixture was stirred vigorously as it was quenched with 50% aqueous sodium hydroxide (˜3 mL, sufficient to provide a granular, white precipitate). The resulting suspension was filtered, and the filtrate was concentrated to give a light brown oil (1-benzyl-7-((R)-α-methylbenzyl)-1,7-diazaspiro[4.4]nonane, 0.90 g, 61%). NMR (CDCl3): 1.4 (d, 3H); 1.6-1.8 (m, 2H); 1.8-2.0 (m, 2H); 2.0-2.15 (m, 1H); 2.35 (d, 1H); 2.4-2.65 (m, 4H); 2.95 (br d, 1H); 3.2 (br dd, 1H); 3.65-3.9 (br dd, 2H).

The above 1-benzyl-7-((R)-α-methylbenzyl)-1,7-diazaspiro[4.4]nonane (0.90 g, 2.8 mmol) was dissolved in methanol (50 mL), and combined with 20% palladium hydroxide on carbon (wet, Degussa type) (0.2 g). The mixture was shaken under hydrogen atmosphere (50 psi) for 3 d, with an additional 0.2 g of catalyst added on day 2. The mixture was filtered through Celite, and the filtrate was concentrated. The residual oil was subjected to Kugelrohr (bulb-to-bulb) distillation (80° C., ˜1 mm Hg pressure) to give a colorless oil (1,7-diazaspiro[4.4]nonane, 300 mg, 84%). This was used directly in the next step without further characterization.

A solution of the above 1,7-diazaspiro[4.4]nonane (150 mg, 1.2 mmol) in dry toluene (5 mL) was purged with nitrogen, and 3-bromopyridine (117 mg, 0.750 mmol), racemic-2,2′-bis(diphenylphosphino)-1,1′-binaphthyl (rac-BINAP) (18 mg, 0.03 mmol), sodium tert-butoxide (100 mg, 1.04 mmol) and tris(dibenzylideneacetone) dipalladium(0) (Pd₂(dba)₃) (14 mg, 0.015 mmol) were added. The mixture was stirred vigorously and heated in an oil bath at 100° C. for 3 h. The mixture was cooled, filtered through diatomaceous earth, and applied to a silica gel column. The column was eluted with a gradient of 0-10% methanol in dichloromethane containing 1% concentrated aqueous ammonium hydroxide. The resulting brown oil (50 mg, 15%) was taken up in methanol and treated with excess 4 M HCl in dioxane (˜1 mL), followed by dilution with ether. The hydrochloride salt initially oiled out, but solidified on standing, and was subsequently triturated with ether. Recrystallization from isopropanol-ether gave a tan solid (7-(3-pyridinyl)-1,7-diazaspiro[4.4]nonane dihydrochloride, 25 mg) with a melting range of 227-232° C. The free base from this material was determined to have a chiral purity of 98%, the major isomer of which is identical by chiral HPLC (Chiralpak AD® column, using 75:25 hexane/ethanol) to S isomer material prepared by other means (e.g., resolution using DTTA salts).

NMR (CD₃OD): 2.2-2.4 (br m, 4H); 2.45-2.65 (br m, 2H); 3.5-3.6 (br m, 2H); 3.6-3.75 (br m, 4H); 7.75-7.9 (br m, 2H); 8.1-8.2 (br m, 2H). MS (M+H)=204.

### (R)-7-(3-pyridinyl)-1,7-diazaspiro[4.4]nonane dihydrochloride

A solution of the less polar diastereomer of N-benzoyl-2-allylproline (R)-α-methylbenzyl amide (4.60 g, 12.6 mmol) in dichloromethane (350 mL), cooled to −78° C., was treated with ozone enriched oxygen for 45 min. The reaction was purged with nitrogen to remove excess ozone and then treated with dimethyl sulfide (1 mL). The reaction was stirred for 2 h and gradually warmed to ambient temperature. The mixture was then treated with triethylsilane (10.5 mL), followed by rapid drop-wise addition of trifluoroacetic acid (7 mL), and stirred overnight under nitrogen atmosphere. The reaction mixture was concentrated to dryness, and the residue dissolved in dichloromethane (100 mL). This solution was washed successively with saturated sodium bicarbonate solution, water and brine (25 mL portions each). The organic layer was dried with anhydrous sodium sulfate, filtered and concentrated. The residue was purified by column chromatography on silica gel with a hexane/ethyl acetate gradient elution (0-50% EtOAc) to give 1.14 g of 1-benzoyl-7-((R)-α-methylbenzyl)-1,7-diazspiro[4.4]nonan-6-one (26%). This lactam (combined with a second lot, produced by the same method; total=2.0 g, 5.8 mmol) in dry tetrahydrofuran (THF) (100 mL) was added drop-wise to an ice cooled suspension of lithium aluminum hydride (0.66 g, 17.3 mmol) in THF (100 mL). After 30 min stirring with ice cooling, the bath was removed and the reaction was heated under reflux overnight. It was then cooled in an ice bath and diluted with ether (100 mL). The reaction was vigorously stirred as it was quenched with 50% aqueous sodium hydroxide (˜3 mL, sufficient to give a granular, white precipitate). The resulting suspension was filtered, and the filtrate was concentrated to give an oil (1-benzyl-7-((R)-α-methylbenzyl)-1,7-diazspiro[4.4]nonane, 1.7 g, 93%). This amine (1.7 g, 5.4 mmol) was dissolved in methanol (200 mL), and combined with 20% palladium hydroxide on carbon (wet, Degussa type) (0.34 g). The mixture was shaken under a hydrogen atmosphere (50 psi) for 3 d, with an additional 0.34 g of catalyst added on day 2. The mixture was filtered through diatomaceous earth, and the filtrate was concentrated. The residual oil was subjected to Kugelrohr (bulb-to-bulb) distillation (80° C., ˜1 mm Hg pressure) to give a colorless oil (1,7-diazaspiro[4.4]nonane, 150 mg, 14%). A solution of this amine (150 mg, 1.2 mmol) in dry toluene (5 mL) was purged with nitrogen, and 3-bromopyridine (117 mg, 0.750 mmol), racemic-2,2′-bis(diphenylphosphino)-1,1′-binaphthyl (rac-BINAP) (18 mg, 0.03 mmol), sodium tert-butoxide (100 mg, 1.04 mmol) and tris(dibenzylideneacetone) dipalladium(0) (Pd₂(dba)₃) (14 mg, 0.015 mmol,) were added. The mixture was stirred vigorously and heated in an oil bath at 100° C. for 3.5 h. The mixture was cooled, filtered through diatomaceous earth, and applied to a silica gel column. The column was eluted with a gradient of 0-10% methanol in dichloromethane containing 1% concentrated aqueous ammonium hydroxide. The resulting brown oil (60 mg, 18%) was taken up in methanol and treated with excess 4 M HCl in dioxane (˜1 mL), followed by dilution with ether. The hydrochloride salt oiled out, but solidified on standing, and was subsequently triturated with ether, then recrystallized from isopropanol-ether to give a tan solid (7-(3-pyridinyl)-1,7-diazaspiro[4.4]nonane dihydrochloride, 40 mg) (melting range 227-233° C.). The free base from this material was determined to have a chiral purity of 94%, the major isomer of which is identical by chiral HPLC (Chiralpak AD® column, using 75:25 hexane/ethanol) to material that had been determined, by x-ray diffraction, to have the R absolute configuration. NMR (CD₃OD): 2.2-2.4 (br m, 4H); 2.45-2.65 (br m, 2H); 3.5-3.6 (br m, 2H); 3.6-3.75 (br m, 4H); 7.75-7.9 (br m, 2H); 8.1-8.2 (br m, 2H). MS (M+H)=204.

### Example 17

### Summary of Salt Formation of 7-(3-pyridinyl)-1,7-diazaspiro[4.4]nonane

Using the techniques described herein, salts of (R)- and (S)-7-(3-pyridinyl)-1,7-diazaspiro[4.4]nonane were prepared. Information regarding the acid used, the equivalents used, the solvents used for recrystallization, and the resulting crystalline or non-crystalline material obtained, are provided in the Tables 6 and 7 below.

TABLE 6
Salt-Forming Acids for the S isomer of 7-(3-pyridinyl)-1,7-diazaspiro[4.4]nonane

| | | Solvents | | | | |
|---|---|---|---|---|---|---|
| Salt Forming | Equiv. | used for | | | | |
| Acid -- | of Acid | Salt | Name of the | | % Yield | |
| Counter Ion | Used | Isolation | Salt Obtained | MP (° C.) | (unoptimized) | Comments |
| Benzoic | 1 | IPAc | Benzoate | 115.5 | 94 | White to off-white |
| | | | | | | solid. May be re- |
| | | | | | | crystallized from IPAc, |
| | | | | | | CH₃CN or acetone |
| | | | | | | (acetone gave a lower |
| | | | | | | yield |
| Citric | 1 | EtOH, | Citrate | | 100 | White, pasty powder |
| | | Acetone, | | | | from IPAc that turned |
| | | MeOH, IPAc | | | | to a gum in a few |
| | | | | | | hours |
| Hydrochloric | 2 | EtOH, | Dihydrochloride | >240 (d) | 90.4 | Light-beige solid |
| | | Acetone | | | | |
| 4- | 1 | IPA | 4- | 136-138 | 70.2 | Off-white solid |
| Hydroxybenzoic | | | Hydroxybenzoate | | | |
| Di-p-toluoyl-D- | 1 | EtOH—H₂O | Di-p-toluoyl-D- | 169.5-170 | 45.0 | White, crystalline solid |
| tartaric | | | tartrate | | | |
| (R)-(−)-Mandelic | 1 | Acetone | (R)-(−)- | 138-149 | 78.6 | White, crystalline solid |
| | | | Mandelate | | | |
| Oxalic | 2 | Acetone | Di-oxalate | 135-138.5 | 82.8 | White solid |
| Phosphoric | 1 | IPA | Phosphate | | | Light-yellow gum |
| Succinic | 1 | EtOH- | Succinate | | | Oily gum |
| | | acetone, | | | | |
| | | EtOAc, | | | | |
| | | MEK | | | | |

TABLE 7
Salt-Forming Acids for the R-isomer of 7-(3-pyridinyl)-1,7-diazaspiro[4.4]nonane

| | | Solvents | | | | |
|---|---|---|---|---|---|---|
| | Equiv. of | used for | | | | |
| Salt Forming Acid -- | Acid | Salt | Name of the | | % Yield | |
| Counter Ion | Used | Isolation | Salt Obtained | MP (° C.) | (unoptimized) | Comments |
| Benzoic | 1 | IPAc | Benzoate | 115.5 | 91.3 | Off-white solid. May be |
| | | | | | | recrystallized from IPAc, CH₃CN |
| | | | | | | or acetone (acetone gave a |
| | | | | | | lower yield for salt formation) |
| 4-Bromobenzoic | 1 | IPAc | 4- | 138-144 | 93.4 | Light-tan solid. May be |
| | | | Bromobenzoate | | | recrystallized from IPAc, |
| | | | | | | acetone or CH₃CN |
| Di-p-toluoyl-L- | 1 | EtOH—H₂O | Di-p-toluoyl-L- | 169-170 | 42.8 | White, crystalline solid |
| tartaric | | | tartrate | | | |
| 4-Hydroxybenzoic | 1 | IPAc-IPA | 4- | 134-135 | 96 | White powder. Acetone is |
| | | | Hydroxybenzoate | | | probably a better solvent for salt |
| | | | | | | formation. Can be re- |
| | | | | | | crystallized from 85% aqueous |
| | | | | | | acetone in good recovery. |
| | | | | | | Source for single crystal X-ray |
| | | | | | | structure (JAM). |
| (R)-(−)-Mandelic | 1 | Acetone | (R)-(−)- | | | Yellow gum |
| acid | | | Mandelate | | | |
| Mucic | 0.5 | MeOH—H₂O | Hemi-mucate | | 60 | White plates |
| Succinic | 1 | EtOH- | Succinate | | | Oily gum |
| | | acetone, | | | | |
| | | MeOH, | | | | |
| | | CH₃CN | | | | |

### Example 18

### Reference Standard Formation and Optical Rotation Determination for (R)-7-(3-Pyridinyl)-1,7-diazaspiro[4,4]nonane p-Hydroxybenzoate

A solution of (R)-7-(3-pyridinyl)-1,7-diazaspiro[4,4]nonane p-hydroxybenzoate (55.82 g, 164 mmol) in absolute ethanol (350 mL) was stirred and heated to reflux temperature, dissolving all solids. Decolorizing carbon (2.88 g) was carefully added, and the mixture was stirred and heated near reflux temperature for 10 min. The hot mixture was filtered over a pad of diatomaceous earth (7.38 g), and the filter cake was washed with hot ethanol (100 mL). The warm filtrate was concentrated via rotary evaporation, and then stirred at room temperature for ˜30 min until precipitation was substantial. Acetone (530 mL) was added rapidly over 7 min, and the mixture was refrigerated at 5° C. for 21 h. The solids were filtered, washed with cold acetone (2×50 mL) and vacuum dried at 50° C. for 22 h. The light-beige solids were transferred to a glass tray, and the large lumps were crushed with a spatula. The material was re-dried under vacuum at 50° C. for 18 h to give 52.3 g (93.7%) of a light-beige, free-flowing powder, mp 136-140.5° C. ¹H NMR spectrum (D₂O) was in agreement with a mono-salt stoichiometry. Purity by achiral HPLC: 99.92%; purity by chiral HPLC: 99.72% % for the shorter retention time isomer; elemental analysis: Calc'd for C₁₂H₁₇N₃.C₇H₆O₃.0.5 H₂O: C, 65.12%; H, 6.90%; N, 11.99%. Found: C, 65.29, 65.17%; H, 6.92, 6.98%; N, 11.96, 11.92% (consistent with a mono-p-hydroxybenzoate hemi-hydrate stoichiometry). ES-MS: [M+H]⁺ at m/e 204 (consistent with the molecular weight of (203.3) of the free base); ¹H NMR (D₂O): δ 7.76 (d, 1H), 7.71 (m, 1H), 7.63 (distorted d, 2H, —C₆H₄— of acid moiety, indicating a mono-salt stoichiometry), 7.16 (dd, 1H), 6.90 (m, 1H), 6.73 (distorted d, 2H, —C₆H₄— of acid moiety, indicating a mono-salt stoichiometry), 3.53 and 3.20 (AB q, 2H), 3.31 (m, 4H), 2.24 (m, 2H), 2.03 (m, 4H); [α]_{D}²⁰−117° (c=10 mg/mL methanol).

A sample of (R)-7-(3-pyridinyl)-1,7-diazaspiro[4,4]nonane was liberated from its p-hydroxybenzoic acid salt (0.76 g, 2.24 mmol) by basification of the salt with 3N sodium hydroxide (15 mL) and extraction with chloroform (4×10 mL). The combined chloroform extracts were washed with water (10 mL) and dried over sodium sulfate. Following filtration, the chloroform was removed by rotary evaporation. The resulting light-yellow oil was further processed by dissolution in chloroform, drying the chloroform solution with sodium sulfate, filtration and concentration by rotary evaporation. The resulting material was dried under vacuum at ˜70° C. for 2.5 h to give 0.44 g (96.9% of (R)-7-(3-pyridinyl)-1,7-diazaspiro[4,4]nonane as a light-yellow oil, [α]_{D}²⁰−54° (c=10 mg/mL methanol).

### Example 19

### Reference Standard Formation and Optical Rotation Determination for (S)-7-(3-Pyridinyl)-1,7-diazaspiro[4,4]nonane p-Hydroxybenzoate

A solution of (S)-7-(3-pyridinyl)-1,7-diazaspiro[4,4]nonane p-hydroxybenzoate (55.4 g, 162 mmol) in absolute ethanol (350 mL) was stirred and heated to reflux temperature, dissolving all solids. Decolorizing carbon (2.81 g) was added, and the mixture was stirred and heated near reflux temperature for 10 min. The hot mixture was filtered over a pad of diatomaceous earth (7.28 g), and the filter cake was washed with hot ethanol (100 mL). Crystallization commenced soon afterwards, and the mixture of off-white solids was stirred for 4-5 h while cooling to room temperature. The mixture was then concentrated via rotary evaporation at 40° C. (water bath), producing 71.24 g of an off-white, yellowish paste. Absolute ethanol (35 mL) was added to the batch. Acetone (635 mL) was added to the flask, and the mixture was stirred and heated to reflux. The heat source was removed and the batch was cooled to room temperature with stirring, then refrigerated at 5° C. for 13 h. The resulting solids were filtered, washed with cold acetone (2×50 mL) and vacuum dried at 50° C. for 6 h. The light-beige solids were transferred to a glass tray, and the large lumps were crushed with a spatula. The material was re-dried under vacuum at 50° C. for 2.5 h to give 54.34 g (97.9%) of a cream colored, lumpy powder, mp 138.5-140.5° C. ¹H NMR spectrum (D₂O) was in agreement with a mono-salt stoichiometry. Purity by achiral HPLC: 99.73%; purity by chiral HPLC: 99.81% for the longer retention time isomer; elemental analysis: Calc'd for C₁₂H₁₇N₃.C₇H₆O₃.0.5 H₂O: C, 65.12%; H, 6.90%; N, 11.99%. Found: C, 65.35, 65.21%; H, 6.96, 6.94%; N, 12.09, 11.98% (consistent with a mono-p-hydroxybenzoate hemi-hydrate stoichiometry). ES-MS: [M+H]⁺ at m/e 204 (consistent with the molecular weight of (203.3) of the free base); ¹H NMR (D₂O): δ 7.76 (d, 1H), 7.72 (m, 1H), 7.62 (distorted d, 2H, —C₆H₄— of acid moiety, indicating a mono-salt stoichiometry), 7.16 (dd, 1H), 6.90 (m, 1H), 6.72 (distorted d, 2H, —C₆H₄— of acid moiety, indicating a mono-salt stoichiometry), 3.53 and 3.20 (AB q, 2H), 3.31 (m, 4H), 2.23 (m, 2H), 2.10 (m, 4H); [α]_{D}²°+121° (c=10 mg/mL methanol)

A sample of (S)-7-(3-pyridinyl)-1,7-diazaspiro[4,4]nonane was liberated from its p-hydroxybenzoic acid salt (0.77 g, 2.25 mmol) by basification of the salt with 3N sodium hydroxide (15 mL) and extraction with chloroform (4×10 mL). The combined chloroform extracts were washed with water (10 mL) and dried over sodium sulfate. Following filtration, the chloroform was removed by rotary evaporation. The resulting light-yellow oil was further processed by dissolution in chloroform, drying the chloroform solution with sodium sulfate, filtration and concentration by rotary evaporation. The resulting material was dried under vacuum at ˜70° C. for 2 h to give 0.44 g (97.3% of (S)-7-(3-pyridinyl)-1,7-diazaspiro[4,4]nonane as a light-yellow oil, [α]_{D}²⁰+55° (c=10 mg/mL methanol).

### Example 20

### DVS Analysis of (R)-7-(3-pyridinyl)-1,7-diazaspiro[4.4]nonane mono-p-hydroxybenzoate

In a dynamic vapor sorption (DVS) apparatus, a sample of (R)-7-(3-pyridinyl)-1,7-diazaspiro[4.4]nonane mono-p-hydroxybenzoate (˜14.1 mg) was subjected to gradually increasing, followed by then gradually decreasing, humidity over a period of about 10 h (see details below). The results, shown in Table 6, indicated that this salt is particularly stable to high humidity, gaining less than 0.2 wt % during the course of the study and readily losing the absorbed moisture as the humidity was decreased. Given its relatively high melting point and crystalline nature, it is therefore a particularly good candidate for drug development.

TABLE 6

| Experiment | Step Isotherm | |
|---|---|---|
| Operator | vti | |
| | (R)-7-(3-pyridinyl)-1,7-diazaspiro[4.4]nonane | |
| Sample Name | p-hydroxybenzoate | |
| Sample Lot # | JAM-022990 | |
| Notes | Prepared from the L-DTTA salt | |
| Drying Temp | 50° | C. |
| Heating Rate | 5° | C./min |
| Max Drying Time | 60 | min |
| Equil Crit | 0.0100 wt % in 2.00 min | |
| Expt Temp | 25° | C. |
| Max Equil Time | 180 | min |

| | |
|---|---|
| Equil Crit | 0.0100 wt % in 5.00 min |
| RH Steps | 5 to 95 to 1 by 5 |
| Data Logging Interval | 2.00 min or 0.0100 wt % |
| Expt Started | Jan. 4, 2007 |
| Run Started | 16:09:22 |

| Elap | Weight | Weight | Samp | Samp |
|---|---|---|---|---|
| Time min | mg | % chg | Temp deg C. | RH % |
| 0.1 | 14.0883 | 0.000 | 25.01 | 0.95 |
| 0.2 | 14.0956 | 0.052 | 25.01 | 0.95 |
| 0.7 | 14.0986 | 0.073 | 25.02 | 0.95 |
| 1.2 | 14.1003 | 0.085 | 25.06 | 0.94 |
| 1.7 | 14.1023 | 0.099 | 25.26 | 0.92 |
| 3.7 | 14.1013 | 0.092 | 27.27 | 0.82 |
| 4.7 | 14.0997 | 0.081 | 28.36 | 0.77 |
| 5.7 | 14.0973 | 0.064 | 29.84 | 0.71 |
| 6.7 | 14.0951 | 0.048 | 31.56 | 0.65 |
| 8.2 | 14.0933 | 0.035 | 34.27 | 0.56 |
| 10.2 | 14.0926 | 0.031 | 37.82 | 0.46 |
| 12.2 | 14.0936 | 0.038 | 41.26 | 0.39 |
| 14.2 | 14.0949 | 0.047 | 44.61 | 0.33 |
| 16.2 | 14.0944 | 0.043 | 46.27 | 0.30 |
| 18.2 | 14.0944 | 0.043 | 45.75 | 0.31 |
| 19.7 | 14.0964 | 0.057 | 44.57 | 0.33 |
| 20.7 | 14.0982 | 0.070 | 43.71 | 0.35 |
| 21.7 | 14.1002 | 0.085 | 42.86 | 0.36 |
| 23.7 | 14.1016 | 0.095 | 41.18 | 0.40 |
| 25.7 | 14.1018 | 0.096 | 39.52 | 0.44 |
| 27.7 | 14.1011 | 0.091 | 37.91 | 0.48 |
| 29.7 | 14.1001 | 0.084 | 36.33 | 0.52 |
| 31.7 | 14.0997 | 0.081 | 34.78 | 0.57 |
| 33.7 | 14.0993 | 0.078 | 33.28 | 0.62 |
| 35.7 | 14.0993 | 0.078 | 31.82 | 0.67 |
| 37.7 | 14.0992 | 0.078 | 30.38 | 0.73 |
| 39.7 | 14.0993 | 0.078 | 28.99 | 0.79 |
| 41.7 | 14.0986 | 0.073 | 27.62 | 1.53 |
| 43.7 | 14.0979 | 0.068 | 26.29 | 4.14 |
| 45.7 | 14.0985 | 0.072 | 25.36 | 4.54 |
| 47.7 | 14.0990 | 0.076 | 25.27 | 5.33 |
| 49.7 | 14.0993 | 0.078 | 25.23 | 5.33 |
| 51.7 | 14.0995 | 0.079 | 25.18 | 5.05 |
| 53.7 | 14.1006 | 0.087 | 25.18 | 4.89 |
| 55.7 | 14.1004 | 0.086 | 25.18 | 5.17 |
| 57.7 | 14.0996 | 0.081 | 25.27 | 9.36 |
| 59.7 | 14.0994 | 0.079 | 25.42 | 9.07 |
| 61.7 | 14.0991 | 0.077 | 25.55 | 9.78 |
| 63.7 | 14.0992 | 0.077 | 25.54 | 9.79 |
| 65.2 | 14.1006 | 0.087 | 25.02 | 10.10 |
| 67.2 | 14.1003 | 0.085 | 25.05 | 10.10 |
| 69.2 | 14.1000 | 0.083 | 25.09 | 12.12 |
| 71.2 | 14.0998 | 0.082 | 25.18 | 14.66 |
| 73.2 | 14.1000 | 0.083 | 25.18 | 14.72 |
| 75.2 | 14.1002 | 0.085 | 25.16 | 14.74 |
| 77.2 | 14.1003 | 0.085 | 25.14 | 14.76 |
| 79.2 | 14.1006 | 0.087 | 25.14 | 18.58 |
| 81.2 | 14.1008 | 0.089 | 25.14 | 19.67 |
| 83.2 | 14.1010 | 0.090 | 25.14 | 19.90 |
| 85.3 | 14.1014 | 0.093 | 25.14 | 19.92 |
| 87.3 | 14.1014 | 0.093 | 25.14 | 19.92 |
| 89.3 | 14.1010 | 0.090 | 25.14 | 23.53 |
| 91.3 | 14.1016 | 0.094 | 25.14 | 24.64 |
| 93.3 | 14.1015 | 0.094 | 25.14 | 24.85 |
| 95.3 | 14.1016 | 0.094 | 25.14 | 24.98 |
| 97.3 | 14.1016 | 0.095 | 25.14 | 24.88 |
| 99.3 | 14.1019 | 0.097 | 25.14 | 24.85 |
| 101.3 | 14.1018 | 0.096 | 25.15 | 28.39 |
| 103.3 | 14.1024 | 0.100 | 25.14 | 29.61 |
| 105.3 | 14.1027 | 0.102 | 25.14 | 29.82 |
| 107.3 | 14.1027 | 0.102 | 25.14 | 29.81 |
| 109.3 | 14.1028 | 0.103 | 25.14 | 29.81 |
| 111.3 | 14.1026 | 0.102 | 25.14 | 33.39 |
| 113.3 | 14.1031 | 0.105 | 25.14 | 34.52 |
| 115.3 | 14.1024 | 0.100 | 25.31 | 34.63 |
| 117.3 | 14.1024 | 0.100 | 25.43 | 34.68 |
| 119.3 | 14.1024 | 0.100 | 25.53 | 34.73 |
| 120.8 | 14.1039 | 0.110 | 25.31 | 35.09 |
| 122.8 | 14.1047 | 0.116 | 25.00 | 35.48 |
| 124.8 | 14.1043 | 0.114 | 25.09 | 34.96 |
| 126.8 | 14.1036 | 0.109 | 25.18 | 34.76 |
| 128.8 | 14.1030 | 0.104 | 25.38 | 34.54 |
| 130.9 | 14.1027 | 0.102 | 25.46 | 35.83 |
| 132.9 | 14.1041 | 0.112 | 25.31 | 39.08 |
| 134.9 | 14.1049 | 0.117 | 25.09 | 40.16 |
| 136.9 | 14.1047 | 0.116 | 25.14 | 39.99 |
| 138.9 | 14.1049 | 0.118 | 25.14 | 39.99 |
| 140.9 | 14.1041 | 0.112 | 25.28 | 39.76 |
| 142.9 | 14.1034 | 0.107 | 25.37 | 42.91 |
| 144.9 | 14.1039 | 0.111 | 25.46 | 44.09 |
| 145.9 | 14.1056 | 0.123 | 25.21 | 44.79 |
| 147.9 | 14.1059 | 0.125 | 25.05 | 45.14 |
| 149.9 | 14.1059 | 0.125 | 25.09 | 45.00 |
| 151.9 | 14.1054 | 0.122 | 25.18 | 45.98 |
| 153.9 | 14.1057 | 0.123 | 25.15 | 48.86 |
| 155.9 | 14.1056 | 0.123 | 25.20 | 49.41 |
| 157.9 | 14.1052 | 0.120 | 25.34 | 49.47 |
| 159.9 | 14.1051 | 0.119 | 25.41 | 49.57 |
| 161.9 | 14.1067 | 0.131 | 25.18 | 50.43 |
| 163.9 | 14.1070 | 0.133 | 25.05 | 50.61 |
| 165.9 | 14.1068 | 0.131 | 25.09 | 50.15 |
| 167.9 | 14.1066 | 0.130 | 25.16 | 49.96 |
| 169.9 | 14.1065 | 0.129 | 25.14 | 49.98 |
| 171.9 | 14.1053 | 0.121 | 25.25 | 52.69 |
| 173.9 | 14.1057 | 0.124 | 25.37 | 53.88 |
| 175.9 | 14.1058 | 0.124 | 25.45 | 54.33 |
| 177.4 | 14.1073 | 0.135 | 25.23 | 55.22 |
| 179.4 | 14.1079 | 0.139 | 25.04 | 55.65 |
| 181.4 | 14.1078 | 0.138 | 25.09 | 55.19 |
| 183.4 | 14.1079 | 0.139 | 25.10 | 55.06 |
| 185.4 | 14.1075 | 0.136 | 25.20 | 54.76 |
| 187.4 | 14.1070 | 0.133 | 25.36 | 54.60 |
| 189.4 | 14.1065 | 0.129 | 25.43 | 57.30 |
| 191.4 | 14.1083 | 0.142 | 25.22 | 59.79 |
| 193.4 | 14.1088 | 0.146 | 25.09 | 60.11 |
| 195.4 | 14.1088 | 0.146 | 25.09 | 60.10 |
| 197.4 | 14.1086 | 0.144 | 25.18 | 59.82 |
| 199.5 | 14.1088 | 0.145 | 25.19 | 59.79 |
| 201.5 | 14.1079 | 0.139 | 25.34 | 62.58 |
| 203.5 | 14.1083 | 0.142 | 25.41 | 63.68 |
| 205.5 | 14.1099 | 0.153 | 25.24 | 65.13 |
| 207.5 | 14.1104 | 0.157 | 25.05 | 65.53 |
| 209.5 | 14.1102 | 0.155 | 25.09 | 65.15 |
| 211.5 | 14.1102 | 0.155 | 25.18 | 64.78 |
| 213.5 | 14.1104 | 0.157 | 25.15 | 64.90 |
| 215.5 | 14.1105 | 0.157 | 25.14 | 66.16 |
| 217.5 | 14.1105 | 0.157 | 25.14 | 68.80 |
| 219.5 | 14.1109 | 0.160 | 25.14 | 69.48 |
| 221.5 | 14.1111 | 0.162 | 25.14 | 69.78 |
| 223.5 | 14.1113 | 0.163 | 25.14 | 69.88 |
| 225.5 | 14.1114 | 0.164 | 25.14 | 69.87 |
| 227.5 | 14.1116 | 0.165 | 25.14 | 69.86 |
| 229.5 | 14.1113 | 0.163 | 25.14 | 72.87 |
| 231.5 | 14.1119 | 0.167 | 25.14 | 74.19 |
| 233.5 | 14.1121 | 0.169 | 25.14 | 74.59 |
| 235.5 | 14.1126 | 0.172 | 25.14 | 74.62 |
| 237.5 | 14.1119 | 0.168 | 25.31 | 74.24 |
| 239.5 | 14.1118 | 0.167 | 25.41 | 75.49 |
| 241.5 | 14.1119 | 0.168 | 25.48 | 77.88 |
| 242.5 | 14.1134 | 0.178 | 25.19 | 79.96 |
| 244.5 | 14.1138 | 0.181 | 25.03 | 80.50 |
| 246.5 | 14.1138 | 0.181 | 25.09 | 80.23 |
| 248.5 | 14.1138 | 0.181 | 25.17 | 79.86 |
| 250.5 | 14.1136 | 0.180 | 25.17 | 81.02 |
| 252.5 | 14.1135 | 0.179 | 25.14 | 83.59 |
| 254.5 | 14.1137 | 0.180 | 25.14 | 84.43 |
| 256.5 | 14.1137 | 0.181 | 25.14 | 84.59 |
| 258.5 | 14.1138 | 0.181 | 25.14 | 84.61 |
| 260.5 | 14.1131 | 0.176 | 25.25 | 84.23 |
| 262.5 | 14.1119 | 0.168 | 25.37 | 86.44 |
| 264.5 | 14.1118 | 0.167 | 25.46 | 88.02 |
| 266.5 | 14.1131 | 0.176 | 25.11 | 90.94 |
| 268.5 | 14.1121 | 0.169 | 25.04 | 90.75 |
| 270.5 | 14.1119 | 0.168 | 25.09 | 90.04 |
| 272.5 | 14.1112 | 0.163 | 25.20 | 89.43 |
| 274.5 | 14.1104 | 0.157 | 25.36 | 88.83 |
| 276.5 | 14.1097 | 0.152 | 25.41 | 89.24 |
| 278.6 | 14.1093 | 0.149 | 25.21 | 93.29 |
| 279.6 | 14.1078 | 0.139 | 25.05 | 94.25 |
| 280.6 | 14.1062 | 0.127 | 25.09 | 94.58 |
| 282.6 | 14.1046 | 0.115 | 25.09 | 94.54 |
| 284.6 | 14.1038 | 0.110 | 25.18 | 94.19 |
| 286.6 | 14.1034 | 0.107 | 25.14 | 94.82 |
| 288.1 | 14.1018 | 0.096 | 25.26 | 94.29 |
| 290.1 | 14.1020 | 0.098 | 25.37 | 94.08 |
| 292.1 | 14.1021 | 0.098 | 25.45 | 93.45 |
| 293.1 | 14.1041 | 0.113 | 25.28 | 92.67 |
| 295.1 | 14.1048 | 0.117 | 25.05 | 92.08 |
| 297.1 | 14.1059 | 0.125 | 25.09 | 90.61 |
| 299.1 | 14.1056 | 0.123 | 25.14 | 89.98 |
| 301.1 | 14.1048 | 0.117 | 25.15 | 89.92 |
| 303.1 | 14.1040 | 0.111 | 25.14 | 89.97 |
| 305.1 | 14.1032 | 0.106 | 25.14 | 89.98 |
| 307.1 | 14.1026 | 0.101 | 25.16 | 89.87 |
| 309.1 | 14.1019 | 0.097 | 25.32 | 89.36 |
| 311.1 | 14.1010 | 0.090 | 25.41 | 86.72 |
| 313.1 | 14.1023 | 0.099 | 25.32 | 86.19 |
| 315.1 | 14.1022 | 0.098 | 25.01 | 86.51 |
| 317.1 | 14.1017 | 0.095 | 25.06 | 85.51 |
| 319.1 | 14.1011 | 0.091 | 25.10 | 85.10 |
| 321.1 | 14.1005 | 0.087 | 25.18 | 84.66 |
| 323.1 | 14.1002 | 0.084 | 25.17 | 84.72 |
| 325.1 | 14.0999 | 0.082 | 25.14 | 82.10 |
| 327.1 | 14.0991 | 0.076 | 25.30 | 80.16 |
| 329.1 | 14.0985 | 0.072 | 25.39 | 79.76 |
| 331.1 | 14.0982 | 0.070 | 25.42 | 79.61 |
| 333.2 | 14.0986 | 0.073 | 25.00 | 78.05 |
| 335.2 | 14.0986 | 0.073 | 25.08 | 76.06 |
| 337.2 | 14.0981 | 0.070 | 25.14 | 75.31 |
| 339.2 | 14.0977 | 0.067 | 25.17 | 75.18 |
| 341.2 | 14.0974 | 0.065 | 25.14 | 75.31 |
| 343.2 | 14.0973 | 0.064 | 25.14 | 75.30 |
| 345.2 | 14.0968 | 0.060 | 25.14 | 72.20 |
| 347.2 | 14.0962 | 0.056 | 25.23 | 70.54 |
| 349.2 | 14.0957 | 0.052 | 25.36 | 69.57 |
| 351.2 | 14.0953 | 0.050 | 25.42 | 69.54 |
| 353.2 | 14.0966 | 0.059 | 25.10 | 71.10 |
| 355.2 | 14.0961 | 0.055 | 25.04 | 70.77 |
| 357.2 | 14.0957 | 0.053 | 25.09 | 70.26 |
| 359.2 | 14.0953 | 0.050 | 25.15 | 69.77 |
| 361.2 | 14.0952 | 0.049 | 25.14 | 69.87 |
| 363.2 | 14.0951 | 0.048 | 25.14 | 69.88 |
| 365.2 | 14.0948 | 0.046 | 25.14 | 69.89 |
| 367.2 | 14.0945 | 0.044 | 25.14 | 66.95 |
| 369.2 | 14.0946 | 0.045 | 25.14 | 65.75 |
| 371.2 | 14.0945 | 0.044 | 25.14 | 65.35 |
| 373.2 | 14.0944 | 0.044 | 25.14 | 65.11 |
| 375.2 | 14.0942 | 0.042 | 25.14 | 65.13 |
| 377.2 | 14.0942 | 0.042 | 25.13 | 65.15 |
| 379.2 | 14.0941 | 0.041 | 25.14 | 65.13 |
| 381.2 | 14.0936 | 0.038 | 25.14 | 62.03 |
| 383.2 | 14.0937 | 0.038 | 25.14 | 60.80 |
| 385.2 | 14.0936 | 0.038 | 25.14 | 60.32 |
| 387.2 | 14.0934 | 0.036 | 25.14 | 60.08 |
| 389.2 | 14.0933 | 0.035 | 25.14 | 60.10 |
| 391.2 | 14.0932 | 0.035 | 25.14 | 60.08 |
| 393.2 | 14.0928 | 0.032 | 25.14 | 56.98 |
| 395.2 | 14.0928 | 0.032 | 25.14 | 55.81 |
| 397.2 | 14.0928 | 0.032 | 25.14 | 55.30 |
| 399.2 | 14.0925 | 0.030 | 25.14 | 55.09 |
| 401.2 | 14.0925 | 0.030 | 25.14 | 55.06 |
| 403.2 | 14.0924 | 0.029 | 25.14 | 55.12 |
| 405.3 | 14.0923 | 0.028 | 25.14 | 55.13 |
| 407.3 | 14.0920 | 0.026 | 25.14 | 51.80 |
| 409.3 | 14.0920 | 0.026 | 25.14 | 50.66 |
| 411.3 | 14.0918 | 0.025 | 25.14 | 50.20 |
| 413.3 | 14.0916 | 0.023 | 25.14 | 50.18 |
| 415.3 | 14.0916 | 0.023 | 25.14 | 50.18 |
| 417.3 | 14.0911 | 0.020 | 25.14 | 46.94 |
| 419.3 | 14.0912 | 0.020 | 25.14 | 45.64 |
| 421.3 | 14.0911 | 0.020 | 25.14 | 45.25 |
| 423.3 | 14.0908 | 0.018 | 25.14 | 45.09 |
| 425.3 | 14.0908 | 0.018 | 25.14 | 45.08 |
| 427.3 | 14.0908 | 0.018 | 25.14 | 45.09 |
| 429.3 | 14.0905 | 0.016 | 25.14 | 45.08 |
| 431.3 | 14.0904 | 0.015 | 25.14 | 41.75 |
| 433.3 | 14.0903 | 0.014 | 25.14 | 40.66 |
| 435.3 | 14.0901 | 0.013 | 25.14 | 40.17 |
| 437.3 | 14.0899 | 0.011 | 25.14 | 40.13 |
| 439.3 | 14.0897 | 0.010 | 25.14 | 40.00 |
| 441.3 | 14.0893 | 0.007 | 25.14 | 36.68 |
| 443.3 | 14.0893 | 0.007 | 25.14 | 35.49 |
| 445.3 | 14.0891 | 0.006 | 25.14 | 35.12 |
| 447.3 | 14.0890 | 0.005 | 25.14 | 35.09 |
| 449.3 | 14.0889 | 0.004 | 25.14 | 35.09 |
| 451.3 | 14.0889 | 0.004 | 25.14 | 35.09 |
| 453.3 | 14.0884 | 0.001 | 25.14 | 31.67 |
| 455.3 | 14.0884 | 0.001 | 25.14 | 30.52 |
| 457.3 | 14.0881 | −0.001 | 25.14 | 30.14 |
| 459.3 | 14.0878 | −0.004 | 25.14 | 30.15 |
| 461.3 | 14.0877 | −0.004 | 25.14 | 30.14 |
| 463.3 | 14.0871 | −0.008 | 25.14 | 26.63 |
| 465.3 | 14.0872 | −0.008 | 25.14 | 25.51 |
| 467.3 | 14.0872 | −0.008 | 25.14 | 25.15 |
| 469.3 | 14.0870 | −0.009 | 25.14 | 25.08 |
| 471.3 | 14.0869 | −0.010 | 25.14 | 25.11 |
| 473.4 | 14.0868 | −0.011 | 25.14 | 25.10 |
| 475.4 | 14.0864 | −0.013 | 25.14 | 21.51 |
| 477.4 | 14.0863 | −0.014 | 25.14 | 21.04 |
| 479.4 | 14.0862 | −0.015 | 25.14 | 20.60 |
| 481.4 | 14.0861 | −0.016 | 25.14 | 20.30 |
| 483.4 | 14.0859 | −0.017 | 25.14 | 20.15 |
| 485.4 | 14.0857 | −0.018 | 25.14 | 20.23 |
| 487.4 | 14.0856 | −0.019 | 25.14 | 20.11 |
| 489.4 | 14.0851 | −0.023 | 25.14 | 16.47 |
| 491.4 | 14.0852 | −0.022 | 25.14 | 15.93 |
| 493.4 | 14.0851 | −0.023 | 25.14 | 15.57 |
| 495.4 | 14.0850 | −0.023 | 25.14 | 15.58 |
| 497.4 | 14.0848 | −0.025 | 25.14 | 15.22 |
| 499.4 | 14.0847 | −0.025 | 25.14 | 15.17 |
| 501.4 | 14.0846 | −0.026 | 25.14 | 15.13 |
| 503.4 | 14.0846 | −0.026 | 25.14 | 15.10 |
| 505.4 | 14.0843 | −0.028 | 25.14 | 9.79 |
| 507.4 | 14.0842 | −0.029 | 25.14 | 9.83 |
| 509.4 | 14.0841 | −0.030 | 25.14 | 9.86 |
| 511.4 | 14.0839 | −0.031 | 25.14 | 9.86 |
| 513.4 | 14.0836 | −0.033 | 25.14 | 9.01 |
| 515.4 | 14.0834 | −0.035 | 25.14 | 5.82 |
| 517.4 | 14.0833 | −0.035 | 25.14 | 4.97 |
| 519.4 | 14.0832 | −0.037 | 25.14 | 4.93 |

### Example 21

### Chiral Analytical HPLC Method

The enantiomeric composition and purity of various samples of 7-(3-pyridinyl)-1,7-diazaspiro[4,4]nonane and its resolved enantiomers were determined using the following method. Samples of the free base (7-(3-pyridinyl)-1,7-diazaspiro[4,4]nonane, (R)-7-(3-pyridinyl)-1,7-diazaspiro[4,4]nonane, or (S)-7-(3-pyridinyl)-1,7-diazaspiro[4,4]nonane) were dissolved in ethanol (˜0.65 mg/mL). Aliquots (10 μL) were analyzed by injection onto a Chiralpak AD, 250×4.6 mm column (Chiral Technologies catalog#19025) and elution with 75:25:0.2 hexanes/ethanol/di-n-butylamine at a flow rate of 1.0 mL/min. The column temperature was maintained at 20° C., and the detector was set at 260 nm. Under these conditions, the R enantiomer typically elutes at 8.3 min and the S enantiomer typically elutes at 9.5 min. Minor variations in retention times are seen, especially when analyses are performed on different days.

Test compounds for the experiments described herein were employed in free or salt form.

The specific pharmacological responses observed may vary according to and depending on the particular active compound selected or whether there are present pharmaceutical carriers, as well as the type of formulation and mode of administration employed, and such expected variations or differences in the results are contemplated in accordance with practice of the present invention.

Although specific embodiments of the present invention are herein illustrated and described in detail, the invention is not limited thereto. The above detailed descriptions are provided as exemplary of the present invention and should not be construed as constituting any limitation of the invention. Modifications will be obvious to those skilled in the art, and all modifications that do not depart from the spirit of the invention are intended to be included with the scope of the appended claims.

1. An acid salt of 7-(3-pyridinyl)-1,7-diazaspiro[4.4]nonane, wherein the acid is succinic acid or oxalic acid.

2. The salt of claim 1, wherein the stoichiometry (molar ratio) of 7-(3-pyridinyl)-1,7-diazaspiro[4.4]nonane to the acid is between 1:2 and 2:1.

3. The salt of claim 1, wherein the stoichiometry (molar ratio) of 7-(3-pyridinyl)-1,7-diazaspiro[4.4]nonane to the acid is 1:1.

4. An acid salt of (R)-7-(3-pyridinyl)-1,7-diazaspiro[4.4]nonane, wherein the acid is hydrochloric acid, oxalic acid, (R)-mandelic acid, benzoic acid, p-bromobenzoic acid, p-hydroxybenzoic acid, galactaric (mucic) acid, or (+)-di-O,O′-p-toluoyl-D-tartaric acid.

5. An acid salt of (S)-7-(3-pyridinyl)-1,7-diazaspiro[4.4]nonane, wherein the acid is hydrochloric acid, oxalic acid, (S)-mandelic acid, benzoic acid, p-bromobenzoic acid, p-hydroxybenzoic acid, galactaric (mucic) acid, or (−)-di-O,O′-p-toluoyl-L-tartaric acid.

6. The salt of claim 4, wherein the stoichiometry (molar ratio) of 7-(3-pyridinyl)-1,7-diazaspiro[4.4]nonane to the acid is between 1:2 and 2:1.

7. The salt of claim 4, wherein the stoichiometry (molar ratio) of 7-(3-pyridinyl)-1,7-diazaspiro[4.4]nonane to the acid is 1:1.

8. The salt of claim 7, wherein the acid is p-hydroxybenzoic acid.

9. (R)-7-(3-pyridinyl)-1,7-diazaspiro[4.4]nonane mono-p-hydroxybenzoate.

10. (S)-7-(3-pyridinyl)-1,7-diazaspiro[4.4]nonane mono-p-hydroxybenzoate.

11. (R)-7-(3-pyridinyl)-1,7-diazaspiro[4.4]nonane or a salt thereof substantially free of (S)-7-(3-pyridinyl)-1,7-diazaspiro[4.4]nonane or a salt thereof.

12. An acid salt of (R)-7-(3-pyridinyl)-1,7-diazaspiro[4.4]nonane in substantially crystalline form.

13. A pharmaceutical composition comprising a compound of claim 11, along with one or more pharmaceutically acceptable carrier.

14. A method for treating or preventing a CNS disorder comprising administering to a subject in need thereof an effective amount of a compound of claim 11.

15. (canceled)

16. (canceled)

17. The method of claim 14, wherein the disorder is selected from the group consisting of depression, anxiety, bipolar disorders, mania, premenstrual dysphoria, panic disorders, bulimia, anorexia, generalized anxiety disorder, seasonal affective disorder, major depressive disorder, obsessive compulsive disorder, rage outbursts, oppositional defiant disorder, Tourette's syndrome, autism, drug and alcohol addiction, tobacco addiction, compulsive eating, and obesity.

18. The method of claim 14, wherein the disorder is selected from the group consisting of pre-senile dementia (early onset Alzheimer's disease), senile dementia (dementia of the Alzheimer's type), Alzheimer's disease, Lewy Body dementia, vascular dementia, AIDS dementia complex, HIV-dementia, Parkinsonism including Parkinson's disease, Pick's disease, progressive supranuclear palsy, Huntington's chorea, tardive dyskinesia, hyperkinesia, Creutzfeld-Jakob disease, epilepsy, attention deficit disorder, attention deficit hyperactivity disorder, dyslexia, schizophrenia, schizophreniform disorder, schizoaffective disorder, mild cognitive impairment (MCI) and age-associated memory impairment (AAMI).

19. The method of claim 14, wherein the disorder is substance addiction.

20. A method for treating or preventing pain or inflammation comprising administering to a subject in need thereof an effective amount of a compound of claim 11.

21. (canceled)

22. (canceled)

23. A method of separating isomers of 7-(3-pyridinyl)-1,7-diazaspiro[4.4]nonane comprising:
(i) converting into diastereomeric salts by reaction with one or both of the stereoisomers of a chiral acid,
(ii) isolating the individual diastereomeric salts by fractional crystallization, and
(iii) liberating the free bases from the isolated salts by treatment with base.

24. The method of claim 23, wherein the chiral acid is one or both of (+)-di-O,O′-p-toluoyl-D-tartaric acid and (−)-di-O,O′-p-toluoyl-L-tartaric acid.

25. A method for preparation of (R)- and (S)-7-(3-pyridinyl)-1,7-diazaspiro[4.4]nonane in substantially pure enantiomeric form comprising:
(i) conversion of a suitably N-protected racemic 2-allylproline into a pair of diastereomeric amides by condensation with a pure enantiomer of an amine containing a chiral auxiliary,
(ii) separation of the diastereomers by means of either chromatography or crystallization, and
(iii) completion of the synthesis in such a manner as the chiral auxiliary is cleaved.

26. The method of claim 25, wherein the pair of diastereomeric intermediates is the N-benzoyl-2-allylproline (R)-α-methylbenzyl amides.
